SEQUENCE LISTING (50 sequences)

SEQ ID NO: 1 - RNA, 67 nt, synthetic construct
  misc_feature 1..67: note = tracrRNA
  source 1..67: mol_type = other RNA
agcatagcaagttaaaataaggctagtccgttatcaacttgaaaaagtggcaccgagtcggtgcttt

SEQ ID NO: 2 - RNA, 36 nt, synthetic construct
  misc_feature 1..36: note = IDT_FANCFsite2
  source 1..36: mol_type = other RNA
gctgcagaagggattccatggttttagagctatgct

SEQ ID NO: 3 - RNA, 36 nt, synthetic construct
  misc_feature 1..36: note = IDT_HEKsite4
  source 1..36: mol_type = other RNA
ggcactgcggctggaggtgggttttagagctatgct

SEQ ID NO: 4 - RNA, 36 nt, synthetic construct
  misc_feature 1..36: note = IDT_VEGFAsite2
  source 1..36: mol_type = other RNA
gaccccctccaccccgcctcgttttagagctatgct

SEQ ID NO: 5 - RNA, 36 nt, synthetic construct
  misc_feature 1..36: note = IDT_VEGFAsite3
  source 1..36: mol_type = other RNA
ggtgagtgagtgtgtgcgtggttttagagctatgct

SEQ ID NO: 6 - DNA, 53 bp, synthetic construct
  misc_feature 1..53: note = NGS_FANCFs2_ON-F
  source 1..53: mol_type = other DNA
tcgtcggcagcgtcagatgtgtataagagacagaggtgctgacgtaggtagtg

SEQ ID NO: 7 - DNA, 55 bp, synthetic construct
  misc_feature 1..55: note = NGS_FANCFs2_ON-R
  source 1..55: mol_type = other DNA
gtctcgtgggctcggagatgtgtataagagacagcgtatcatttcgcggatgttc

SEQ ID NO: 8 - DNA, 51 bp, synthetic construct
  misc_feature 1..51: note = NGS_Index_F1
  source 1..51: mol_type = other DNA
aatgatacggcgaccaccgagatctacacctctctattcgtcggcagcgtc

SEQ ID NO: 9 - DNA, 51 bp, synthetic construct
  misc_feature 1..51: note = NGS_Index_F2
  source 1..51: mol_type = other DNA
aatgatacggcgaccaccgagatctacactatcctcttcgtcggcagcgtc

SEQ ID NO: 10 - DNA, 51 bp, synthetic construct
  misc_feature 1..51: note = NGS_Index_F3
  source 1..51: mol_type = other DNA
aatgatacggcgaccaccgagatctacacgtaaggagtcgtcggcagcgtc

SEQ ID NO: 11 - DNA, 51 bp, synthetic construct
  misc_feature 1..51: note = NGS_Index_F4
  source 1..51: mol_type = other DNA
aatgatacggcgaccaccgagatctacacactgcatatcgtcggcagcgtc

SEQ ID NO: 12 - DNA, 51 bp, synthetic construct
  misc_feature 1..51: note = NGS_Index_F5
  source 1..51: mol_type = other DNA
aatgatacggcgaccaccgagatctacacaaggagtatcgtcggcagcgtc

SEQ ID NO: 13 - DNA, 51 bp, synthetic construct
  misc_feature 1..51: note = NGS_Index_F6
  source 1..51: mol_type = other DNA
aatgatacggcgaccaccgagatctacacctaagccttcgtcggcagcgtc

SEQ ID NO: 14 - DNA, 51 bp, synthetic construct
  misc_feature 1..51: note = NGS_Index_F7
  source 1..51: mol_type = other DNA
aatgatacggcgaccaccgagatctacaccgtctaattcgtcggcagcgtc

SEQ ID NO: 15 - DNA, 51 bp, synthetic construct
  misc_feature 1..51: note = NGS_Index_F8
  source 1..51: mol_type = other DNA
aatgatacggcgaccaccgagatctacactctctccgtcgtcggcagcgtc

SEQ ID NO: 16 - DNA, 47 bp, synthetic construct
  misc_feature 1..47: note = NGS_Index_R1
  source 1..47: mol_type = other DNA
caagcagaagacggcatacgagattcgccttagtctcgtgggctcgg

SEQ ID NO: 17 - DNA, 47 bp, synthetic construct
  misc_feature 1..47: note = NGS_Index_R2
  source 1..47: mol_type = other DNA
caagcagaagacggcatacgagatctagtacggtctcgtgggctcgg

SEQ ID NO: 18 - DNA, 47 bp, synthetic construct
  misc_feature 1..47: note = NGS_Index_R3
  source 1..47: mol_type = other DNA
caagcagaagacggcatacgagatttctgcctgtctcgtgggctcgg

SEQ ID NO: 19 - DNA, 47 bp, synthetic construct
  misc_feature 1..47: note = NGS_Index_R4
  source 1..47: mol_type = other DNA
caagcagaagacggcatacgagatgctcaggagtctcgtgggctcgg

SEQ ID NO: 20 - DNA, 47 bp, synthetic construct
  misc_feature 1..47: note = NGS_Index_R5
  source 1..47: mol_type = other DNA
caagcagaagacggcatacgagataggagtccgtctcgtgggctcgg

SEQ ID NO: 21 - DNA, 47 bp, synthetic construct
  misc_feature 1..47: note = NGS_Index_R6
  source 1..47: mol_type = other DNA
caagcagaagacggcatacgagatcatgcctagtctcgtgggctcgg

SEQ ID NO: 22 - DNA, 47 bp, synthetic construct
  misc_feature 1..47: note = NGS_Index_R7
  source 1..47: mol_type = other DNA
caagcagaagacggcatacgagatgtagagaggtctcgtgggctcgg

SEQ ID NO: 23 - DNA, 47 bp, synthetic construct
  misc_feature 1..47: note = NGS_Index_R8
  source 1..47: mol_type = other DNA
caagcagaagacggcatacgagatcagcctcggtctcgtgggctcgg

SEQ ID NO: 24 - DNA, 20 bp, synthetic construct
  misc_feature 1..20: note = MNase_F
  source 1..20: mol_type = other DNA
gatcggaagagcacacgtct

SEQ ID NO: 25 - DNA, 33 bp, synthetic construct
  misc_feature 1..33: note = MNase_R
  source 1..33: mol_type = other DNA
acactctttccctacacgacgctcttccgatct

SEQ ID NO: 26 - DNA, 58 bp, synthetic construct
  misc_feature 1..58: note = PE_i5
  source 1..58: mol_type = other DNA
aatgatacggcgaccaccgagatctacactctttccctacacgacgctcttccgatct

SEQ ID NO: 27 - DNA, 64 bp, synthetic construct
  misc_feature 1..64: note = PE_i701
  source 1..64: mol_type = other DNA
caagcagaagacggcatacgagatcgtgatgtgactggagttcagacgtgtgctcttccgatct

SEQ ID NO: 28 - DNA, 64 bp, synthetic construct
  misc_feature 1..64: note = PE_i702
  source 1..64: mol_type = other DNA
caagcagaagacggcatacgagatacatcggtgactggagttcagacgtgtgctcttccgatct

SEQ ID NO: 29 - DNA, 64 bp, synthetic construct
  misc_feature 1..64: note = PE_i703
  source 1..64: mol_type = other DNA
caagcagaagacggcatacgagatgcctaagtgactggagttcagacgtgtgctcttccgatct

SEQ ID NO: 30 - DNA, 64 bp, synthetic construct
  misc_feature 1..64: note = PE_i703
  source 1..64: mol_type = other DNA
caagcagaagacggcatacgagattggtcagtgactggagttcagacgtgtgctcttccgatct

SEQ ID NO: 31 - DNA, 64 bp, synthetic construct
  misc_feature 1..64: note = PE_i705
  source 1..64: mol_type = other DNA
caagcagaagacggcatacgagatattggcgtgactggagttcagacgtgtgctcttccgatct

SEQ ID NO: 32 - DNA, 64 bp, synthetic construct; identical to SEQ ID NO: 31
  misc_feature 1..64: note = PE_i706
  source 1..64: mol_type = other DNA

SEQ ID NO: 33 - DNA, 64 bp, synthetic construct
  misc_feature 1..64: note = PE_i707
  source 1..64: mol_type = other DNA
caagcagaagacggcatacgagatgatctggtgactggagttcagacgtgtgctcttccgatct

SEQ ID NO: 34 - DNA, 64 bp, synthetic construct
  misc_feature 1..64: note = PE_i708
  source 1..64: mol_type = other DNA
caagcagaagacggcatacgagattcaagtgtgactggagttcagacgtgtgctcttccgatct

SEQ ID NO: 35 - DNA, 64 bp, synthetic construct
  misc_feature 1..64: note = PE_i709
  source 1..64: mol_type = other DNA
caagcagaagacggcatacgagatctgatcgtgactggagttcagacgtgtgctcttccgatct

SEQ ID NO: 36 - DNA, 64 bp, synthetic construct
  misc_feature 1..64: note = PE_i710
  source 1..64: mol_type = other DNA
caagcagaagacggcatacgagataagctagtgactggagttcagacgtgtgctcttccgatct

SEQ ID NO: 37 - DNA, 64 bp, synthetic construct
  misc_feature 1..64: note = PE_i711
  source 1..64: mol_type = other DNA
caagcagaagacggcatacgagatgtagccgtgactggagttcagacgtgtgctcttccgatct

SEQ ID NO: 38 - DNA, 64 bp, synthetic construct
  misc_feature 1..64: note = PE_i712
  source 1..64: mol_type = other DNA
caagcagaagacggcatacgagattacaaggtgactggagttcagacgtgtgctcttccgatct

SEQ ID NO: 39 - DNA, 64 bp, synthetic construct
  misc_feature 1..64: note = PE_i713
  source 1..64: mol_type = other DNA
caagcagaagacggcatacgagatatcagtgtgactggagttcagacgtgtgctcttccgatct

SEQ ID NO: 40 - DNA, 64 bp, synthetic construct
  misc_feature 1..64: note = PE_i714PE_i714PE_i714PE_i714
  source 1..64: mol_type = other DNA
caagcagaagacggcatacgagataggaatgtgactggagttcagacgtgtgctcttccgatct

SEQ ID NO: 41 - DNA, 64 bp, synthetic construct
  misc_feature 1..64: note = PE_i715
  source 1..64: mol_type = other DNA
caagcagaagacggcatacgagatattccggtgactggagttcagacgtgtgctcttccgatct

SEQ ID NO: 42 - DNA, 64 bp, synthetic construct
  misc_feature 1..64: note = PE_i716
  source 1..64: mol_type = other DNA
caagcagaagacggcatacgagatccactcgtgactggagttcagacgtgtgctcttccgatct

SEQ ID NO: 43 - DNA, 64 bp, synthetic construct
  misc_feature 1..64: note = PE_i717
  source 1..64: mol_type = other DNA
caagcagaagacggcatacgagatcgattagtgactggagttcagacgtgtgctcttccgatct

SEQ ID NO: 44 - DNA, 64 bp, synthetic construct
  misc_feature 1..64: note = PE_i718
  source 1..64: mol_type = other DNA
caagcagaagacggcatacgagatcttcgagtgactggagttcagacgtgtgctcttccgatct

SEQ ID NO: 45 - DNA, 64 bp, synthetic construct
  misc_feature 1..64: note = PE_i719
  source 1..64: mol_type = other DNA
caagcagaagacggcatacgagatgaatgagtgactggagttcagacgtgtgctcttccgatct

SEQ ID NO: 46 - DNA, 64 bp, synthetic construct
  misc_feature 1..64: note = PE_i720
  source 1..64: mol_type = other DNA
caagcagaagacggcatacgagatgcggacgtgactggagttcagacgtgtgctcttccgatct

SEQ ID NO: 47 - DNA, 64 bp, synthetic construct
  misc_feature 1..64: note = PE_i721
  source 1..64: mol_type = other DNA
caagcagaagacggcatacgagatggaactgtgactggagttcagacgtgtgctcttccgatct

SEQ ID NO: 48 - DNA, 64 bp, synthetic construct
  misc_feature 1..64: note = PE_i722
  source 1..64: mol_type = other DNA
caagcagaagacggcatacgagattagttggtgactggagttcagacgtgtgctcttccgatct

SEQ ID NO: 49 - DNA, 64 bp, synthetic construct
  misc_feature 1..64: note = PE_i723
  source 1..64: mol_type = other DNA
caagcagaagacggcatacgagattcgggagtgactggagttcagacgtgtgctcttccgatct

SEQ ID NO: 50 - DNA, 64 bp, synthetic construct
  misc_feature 1..64: note = PE_i724
  source 1..64: mol_type = other DNA
caagcagaagacggcatacgagattctgaggtgactggagttcagacgtgtgctcttccgatct

(19) United States
(12) Patent Application Publication
(10) Pub. No.: US 2025/0002991 A1
(43) Pub. Date: Jan. 2, 2025

(54) METHODS AND COMPOSITIONS FOR GENOME EDITING

(71) Applicant: THE JOHNS HOPKINS UNIVERSITY, Baltimore, MD (US)

(72) Inventors: Roger Zou, Baltimore, MD (US); Yang Liu, Baltimore, MD (US); Taekjip Ha, Baltimore, MD (US); Oscar REYES GAIDO, Baltimore, MD (US)

(21) Appl. No.: 18/710,522

(22) PCT Filed: Nov. 15, 2022
(86) PCT No.: PCT/US2022/079887

§ 371 Date: May 15, 2024

Related U.S. Application Data
(60) Provisional application No. 63/279,651, filed on Nov. 15, 2021.

(51) Int. Cl. C12Q 1/6869; C12N 9/22; C12N 15/11; C12N 15/90

(52) U.S. Cl. CPC C12Q 1/6869; C12N 9/22; C12N 15/11; C12N 15/907; C12N 2310/20

(57) ABSTRACT

Compositions and methods for discovery of off-target CRISPR-Cas genome editing in patients, patient-derived cells and animal models are provided.

### CROSS-REFERENCES TO RELATED APPLICATIONS

This application claims the benefit of priority of U.S. Provisional Application No. 63/279,651 filed on Nov. 15, 2021, which is incorporated herein by reference in its entirety and for all purposes.

### STATEMENT REGARDING FEDERALLY SPONSORED RESEARCH

This invention was made with government support under grant nos. DK1 27432 and GM122569 awarded by the National Institutes of Health. The government has certain rights in the invention.

### SEQUENCE LISTING

The contents of the sequence listing text file named “348358_11902_ST25.xml”, which was created on Nov. 8, 2022 and is 11,383 bytes in size, are incorporated herein by reference in its entirety.

### FIELD

Methods for improving off-target sensitivity in genome editing are disclosed. Compositions include inhibitors of DNA repair.

### BACKGROUND

CRISPR-Cas genome editing is a transformative technology that holds great potential for therapeutic breakthroughs. However, unintended off-target mutagenesis raises concerns for safety and applicability, especially for in vivo and therapeutic contexts, so accurate and sensitive methods for discovery of CRISPR off-target editing is essential. However, almost all current off-target discovery methods are limited to purified DNA or restricted cellular systems and are incapable of direct discovery in vivo. Recently, a method called DISCOVER-Seq was the first to demonstrate direct off-target discovery in vivo, but suffers from low sensitivity.

### SUMMARY

Embodiments are directed to compositions and methods for discovery of off-target genome editing in vitro or in vivo.

Accordingly, in certain embodiments a method of determining specificity of a gene editing complex or off-target genome editing in vitro or in vivo, comprises contacting a cell with a gene-editing complex and at least one guide RNA (gRNA) that targets a nucleic acid sequence of interest: administering to a cell an inhibitor of DNA repair: assaying for target nucleic acid sequences comprising one or more nicks. In certain aspects, the gene editing complex comprises clustered regularly interspaced short palindromic repeat (CRISPR) nucleases, zinc-finger nucleases (ZFNs), transcription activator-like effector nucleases (TALENs), meganucleases, Argonaute family of endonucleases, endo- or exo-nucleases, or combinations thereof.

In certain embodiments, a method of detecting off-target genome editing in vitro or in vivo, comprises contacting a cell in vitro or in vivo with a Clustered Regularly Interspaced Short Palindromic Repeat (CRISPR)-associated endonuclease and at least one guide RNA (gRNA) that targets a nucleic acid sequence of interest: administering to a cell in vitro or in vivo an inhibitor of DNA repair, wherein the inhibitor modulates meiotic recombination 11 (MRE11) localization at the target nucleic acid sequence: detecting MRE11 at on- and off-target nucleic acid sequences. In certain aspects, the MRE11 is detected by chromatin immunoprecipitation with sequencing (ChIP-seq) and/or quantified by qPCR. In certain aspects, the inhibitor of DNA repair comprises one or more DNA-dependent protein kinase catalytic subunit (DNA-PKcs) inhibitors. In certain aspects, the one or more DNA-Pkc inhibitors comprise 2-(4-ethylpiperazin-1-yl)-N-[4-(2-morpholin-4-yl-4-oxochromen-8-yl)dibenzothiophen-1-yl]acetamide (Ku-60648), 2-(morpholin-4-yl)benzo (h) chromen-4-one (Nu7026), N-methyl-2-morpholin-4-yl-N-[6-[2-(8-oxa-3-azabicyclo[3.2.1]octan-3-yl)-4-oxochromen-8-yl]dibenzothiophen-2-yl]acetamide (NU5455), 7-methyl-2-[(7-methyl-[1,2,4]triazolo[1,5-a]pyridin-6-yl)amino]-9-(oxan-4-yl) purin-8-one (AZD7648), 2-N-morpholino-8-dibenzofuranyl-chromen-4-one (NU7427), 2-N-morpholino-8-dibenzothiophenyl-chromen-4-one (NU7441), non-coding microRNAs (miRNAs), siRNAs or combinations thereof. In certain aspects, the DNA-Pkc inhibitor is Ku-60648. In certain aspects, the DNA-Pkc inhibitor is Nu7026. In certain aspects, the CRISPR-associated endonuclease is Type I, Type II, or Type III Cas endonuclease. In certain aspects, the CRISPR-associated endonuclease is a Cas9 endonuclease, a Cas12 endonuclease, a CasX endonuclease, or a CasΦ endonuclease. In certain aspects, the CRISPR-associated endonuclease is a Cas9 nuclease. In certain aspects, the Cas9 nuclease is a Staphylococcus aureus Cas9 nuclease.

In certain embodiments, a method of determining specificity of a candidate genome editing complex in vivo, comprises contacting a cell in vitro or in vivo with a Clustered Regularly Interspaced Short Palindromic Repeat (CRISPR)-associated endonuclease and at least one guide RNA (gRNA) that targets a nucleic acid sequence of interest: administering to a cell in vitro or in vivo an inhibitor of DNA repair, wherein the inhibitor modulates meiotic recombination 11 (MRE11) localization at the target nucleic acid sequence: detecting MRE11 at on- and off-target nucleic acid sequences. In certain aspects, the MRE11 is detected by chromatin immunoprecipitation with sequencing (ChIP-seq) and/or quantified by qPCR. In certain aspects, the inhibitor of DNA repair comprises one or more DNA-dependent protein kinase catalytic subunit (DNA-PKcs) inhibitors. In certain aspects, the one or more DNA-Pkc inhibitors comprise 2-(4-ethylpiperazin-1-yl)-N-[4-(2-morpholin-4-yl-4-oxochromen-8-yl)dibenzothiophen-1-yl]acetamide (Ku-60648), 2-(morpholin-4-yl)benzo (h) chromen-4-one (Nu7026), N-methyl-2-morpholin-4-yl-N-[6-[2-(8-oxa-3-azabicyclo[3.2.1]octan-3-yl)-4-oxochromen-8-yl]dibenzothiophen-2-yl]acetamide (NU5455), 7-methyl-2-[(7-methyl-[1,2,4]triazolo[1,5-a]pyridin-6-yl)amino]-9-(oxan-4-yl) purin-8-one (AZD7648), 2-N-morpholino-8-dibenzofuranyl-chromen-4-one (NU7427), 2-N-morpholino-8-dibenzothiophenyl-chromen-4-one (NU7441), non-coding microRNAs (miRNAs), siRNAs or combinations thereof. In certain aspects, the CRISPR-associated endonuclease is Type I, Type II, or Type III Cas endonuclease. In certain aspects, the CRISPR-associated endonuclease is a Cas9 endonuclease, a Cas 12 endonuclease, a CasX endonuclease, or a CasΦ endonuclease. In certain aspects, the CRISPR-associated endonuclease is a Cas9 nuclease.

Other aspects are discussed infra.

### Definitions

The terminology used herein is for the purpose of describing particular embodiments only and is not intended to be limiting of the invention. Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, “DNA repair genes” are those genes which encode proteins involved in DNA repair mechanisms. The term DNA repair genes refers to the genes and also to the proteins they encode.

“Expression vector” refers to a vector comprising a recombinant polynucleotide comprising expression control sequences operatively linked to a nucleotide sequence to be expressed. An expression vector comprises sufficient cis-acting elements for expression: other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “kit” refers to any delivery system for delivering materials. Inclusive of the term “kits” are kits for both research and clinical applications. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides or liposomes. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used in this specification and the appended claims, the term “or” is generally employed in its sense including “and/or” unless the content clearly dictates otherwise.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1a-1j show DNA-PKcs inhibition increases MRE11 enrichment at CRISPR targeted locations.

FIGS. 1a-1b show schematic of genome-wide CRISPR off-target detection using MRE11 ChIP-seq. FIG. 1a: Cells are unsynchronized, so only some cells have MRE11 at Cas9 cut sites at a given time (DISCOVER-Seq). FIG. 1b: Inhibition of NHEJ directs DNA repair to slower, MRE11-dependent pathways.

FIG. 1C: Effect of repair factor inhibition on MRE11 residence at the VEGFA site 3 on-target site, measured by ChIP-qPCR estimating “reads per million” (RPM) enrichment at 12 hours after Cas9 delivery in HEK293T cells. ‘no drug’ means no repair factor inhibition, ‘Olaparib’ is a poly ADP ribose polymerase (PARP) inhibitor, ‘Scr7’ is a DNA Ligase IV inhibitor, ‘Ku-55933’ is an Ataxia-telangiectasia mutated (ATM) serine/threonine kinase inhibitor, and ‘Ku-60648’ and ‘Nu7026’ are DNA-dependent protein kinase catalytic subunit (DNA-PKcs) inhibitors. Samples with DNA-PKcs inhibition (n=4) have significantly higher estimated RPM compared to samples without DNA-PKcs inhibition (n=8) using Student's t-test (p<1E-4).

FIG. 1d: Increased MRE11 residence upon DNA-PKcs inhibition using Ku-60648 (black, top graph) versus without inhibition (grey, bottom graph), measured by ChIP-qPCR. Measured (left plot) over multiple time points (4 h, 12 h, 24 h) after delivery of Cas9 targeting VEGFA site 2 in HEK293T, (middle plot) with Cas9 targeting FANCF site 2 in K562 at 12 h, and (right plot) with Cas9 targeting HEK site 4 in HEK293T at 12 h. Plots display the mean over two biological replicates for left and middle plots, and one replicate for the right plot.

FIG. 1e: Plot of estimated RPM enrichment normalized to the no drug sample from data in panel d, for sample pairs with (‘Ku-60648’) or without (′no drug) DNA-PKcs inhibition. Normalized RPM enrichments with DNA-PKcs inhibition was significantly higher than without inhibitor (p=0.0001), using Wilcoxon matched-pairs signed rank test, n=14 biological replicates.

FIG. 1f: The proportion of 53BP1 foci relative to BRCA1 as detected by SIM in cells exposed to Cas9 targeting a multi-target gRNA with 126 genome-wide target sites.

FIG. 1g: The number of repair foci (53BP1 or BRCA1) as detected by SIM in cells with or without Cas9 (‘+Cas9’ or ‘−Cas9’, respectively), with or without Ku-60648 (‘KU’ vs ‘nD’, respectively), targeting 126 genome-wide sites with a multi-target gRNA.

FIGS. 1h-1i: Representative images corresponding to FIG. 1g.

FIG. 1j: Histogram of insertion-deletion mutations (indels) or no mutations (‘0’) at 48 hours after Cas9 editing of ACTB, either without DNA-PKcs inhibitor (‘Cas9, no inhibitor’) or with inhibitor (‘Cas9, Ku-60648’). Untreated cells not exposed to Cas9 is shown for reference (‘Untreated (no Cas9)’).

FIGS. 2a-2m show DNA-PKcs inhibition increases the sensitivity of CRISPR off-target detection.

FIGS. 2a-2b: Plots of MRE11 ChIP-seq enrichment within a 1.5 kb window for samples with (y-axis) or without (x-axis) DNA-PKcs inhibition at all (a) FANCF site 2 or (b) VEGFA site 2 Cas9 target sites in K562 detected from the DNA-PKcs inhibited samples. Significant differences (p<1E-3 or p<1E-5) between y-axis and x-axis values were determined using Wilcoxon rank sum test.

FIGS. 2c-2d: Genome browser visualization of MRE11 enrichment at an (c) on-target and (d) representative off-target position from K562 with Cas9 targeting FANCF site 2, with (black) or without (grey) DNA-PKcs inhibition.

FIGS. 2e-2f: Same as FIGS. 2a and 2b, at positions 10 kb downstream from the actual cut site, to measure background enrichment adjacent to cut sites. MRE11 enrichment with (y-axis) versus without (x-axis) DNA-PKcs inhibition at the adjacent background locations was not significantly different (p=0.21 or 0.18), determined using Wilcoxon rank sum test.

FIG. 2g: Number of discovered off-target sites with (black, right bar) or without (dark grey, left bar) DNA-PKcs inhibition for VEGFA site 2. Quantification of FIG. 5b.

FIGS. 2h-2i: Number of discovered off-target sites with (black, right bars) or without (grey, left bars) DNA-PKcs inhibition for (h) VEGFA site 3. FANCF site 2, and (i) HEK site 4 gRNAs. Quantification of FIG. 5c.

FIG. 2j: Overlap in the identity of Cas9 target sites discovered from samples with DNA-PKcs inhibition (‘DNA-PKi only’: left circle), without DNA-PKcs inhibition (‘no drug only’: right circle), or found in both samples (‘both’: overlapping area). 4 gRNAs were evaluated.

FIG. 2k: (left plot) Measurement of insertion-deletion mutations (indels) at the on-target and sole off-target site (OFF0) discovered by the original DISCOVER-Seq, for K562 cells with or without Cas9 with the FANCF site 2 gRNA (‘+Cas9’ or ‘-Cas9’, respectively). (right plot) Measurement of indels at off-target sites exclusively discovered by DISCOVER-Seq+. Plots display mean of 3 biological replicates, error bars represent±1 standard deviation from mean. n.s. indicates not significant, * indicates p<0.05, ** indicates p<0.01, and *** indicates p<0.001 using Student's t-test.

FIG. 2l: Overlap in the identity of target sites identified by DISCOVER-Seq+versus GUIDE-seq.

FIG. 2m: For the 15 target sites (1 on-target, 14 off-targets) of the FANCF site 2 gRNA identified by DISCOVER-Seq+ (‘DSeq+’), the chart shows which sites are also identified by DISCOVER-Seq alone (‘+’ under ‘DSeq’), which sites have indels detectable by targeted deep sequencing (‘+’ under ‘Indels’), and which sites were also detectable by GUIDE-seq (‘+’ under ‘Gseq’). Target sites labeled with ‘N/A’ under ‘Indels’ were unable to be successfully amplified by PCR for targeted sequencing.

FIGS. 3a-3j show DISCOVER-Seq+ in human iPSCs and primary T-cells. FIG. 3a: VEGFA site 2 Cas9 target sites detected using DISCOVER-Seq (left) versus DISCOVER-Seq+ (right) in WTC-11 iPSCs.

FIGS. 3b-3c: Genome browser visualization of MRE11 enrichment at an (b) on-target and (c) representative off-target position with 4 mismatches (‘4 mm’) in WTC-11 iPSCs with Cas9 targeting VEGFA site 2. DISCOVER-Seq+data in black (with Ku-60648, bottom graph), DISCOVER-Seq data in grey (with no drug exposure, bottom graph).

FIG. 3d: Schematic of the DISCOVER-Seq+protocol in the knock-in of a Chimeric Antigen Receptor (CAR) into the TRA locus of primary human T-cells.

FIG. 3e: TRA Cas9 target sites in primary T-cells detected using DISCOVER-Seq (left) versus DISCOVER-Seq+ (right).

FIGS. 3f-3g: Genome browser visualization of MRE11 enrichment at two representative 4-mismatch (‘4 mm’) off-target positions in primary human T-cells with Cas9 targeting TRA for knock-in of a Chimeric Antigen Receptor (CAR) template. DISCOVER-Seq+data in black (with Ku-60648, top graph), DISCOVER-Seq data in grey (with no drug exposure, bottom graph).

FIG. 3h: Plots of MRE11 ChIP-seq enrichment within a 1.5 kb window for samples with (y-axis) or without (x-axis) DNA-PKcs inhibition at all TRA Cas9 off-target sites in primary human T-cells from the DNA-PKcs inhibited samples. Significant differences (p<1E-3 or p<1E-5) between y-axis and x-axis values were determined using Wilcoxon rank sum test.

FIG. 3i: Same as panel h, for cells delivered with Cas9 but without gRNA (negative control).

FIG. 3j: TRA Cas12a (Cpf1) target sites in primary T-cells. The results were same between DISCOVER-Seq and DISCOVER-Seq+: only the on-target site was detected.

FIGS. 4a-4g are DISCOVER-Seq+ in mice.

FIG. 4a: Schematic of DISCOVER-Seq+protocol in mice.

FIGS. 4b-4d: Genome browser visualization of MRE11 enrichment at the (b) PCSK9 on-target site (‘ON-target’) and (c,d) two off-target sites (‘OFF-target’) with 2 mismatches each (‘2 mm’) in the liver of mice transduced with adenovirus expressing Cas9 targeting PCSK9. Mice were dosed twice a day (b.i.d.) with either 25 mg/kg Ku-60648 (‘Ku-60648’); black, top graph) or with vehicle (‘no drug’; grey, bottom graph). FIG. 4e: Number of detected genome-wide target sites in the mouse genome mm 10 with Cas9/gRNA targeting PCSK9, identified using DISCOVER-Seq (DSeq) versus DISCOVER-Seq+ (DSeq+). N=5 biological replicates (mice) were used for each condition: DISCOVER-Seq+detected significantly more target sites than DISCOVER-Seq, using Student's paired t-test (p=0.0223).

FIG. 4f: Overlap in the mouse PCSK9 target sites identified by in vivo DISCOVER-Seq+ in this work—‘DSeq+ (this work)’, in vivo DISCOVER-Seq in this work—‘DSeq (this work)’, and the original DISCOVER-Seq manuscript—‘DSeq (Wienert et al.)’.

FIG. 4g: PCSK9 Cas9 target sites detected using DISCOVER-Seq (left) versus DISCOVER-Seq+ (right) in mouse liver. Off-target detection for each condition was performed on sequencing data pooled across 5 biological mouse replicates.

FIG. 5a-5b show lists of off-target sites discovered by MRE11 ChIP-seq with DNA-PKcs inhibition.

FIG. 5a: VEGFA site 2 Cas9 target sites detected using DISCOVER-Seq (left) versus DISCOVER-Seq+ (right) in K562 cells.

FIG. 5b: FANCF site 2. VEGFA site 3, or HEK site 4 Cas9 target sites detected using DISCOVER-Seq versus DISCOVER-Seq+ in K562 or HEK293T cells, at 4 h, 12 h, or 24 h after Cas9 delivery.

FIGS. 6a-6f show Effect of DNA-PKcs inhibition on DNA repair and MRE11 enrichment at target sites.

FIGS. 6a-6b: Histogram of insertion-deletion mutations (indels) at 48 hours after Cas9 editing of FANCF site 2 at the on-target site, either (a) without DNA-PKcs inhibitor (‘no inhibitor’) or (b) with inhibitor (‘Ku-60648’).

FIGS. 6c-6d: Same as FIGS. 6a-6b, at the select off-target site ‘OFF0’.

FIG. 6e: Plot of MRE11 ChIP-seq enrichment at all DISCOVER-Seq+detected target sites within a 1.5 kb window for Cas9 targeting VEGFA site 2 in WTC-11 iPSCs. MRE11 enrichment from DISCOVER-Seq+data (y-axis) versus DISCOVER-Seq data (x-axis) was significantly different (p<1E-5), determined using the Wilcoxon rank sum test.

FIG. 6f: Plot of MRE11 ChIP-seq enrichment at all DISCOVER-Seq+detected target sites within a 1.5 kb window for Cas9 targeting PCSK9 in the liver of mice. MRE11 enrichment from DISCOVER-Seq+data (y-axis) versus DISCOVER-Seq data (x-axis) was significantly different (p<1E-4), determined using the Wilcoxon rank sum test.

### DETAILED DESCRIPTION

Genome editing involves the targeted induction of DNA double strand breaks (DSBs) by a CRISPR-associated endonuclease such as Streptococcus pyogenes Cas9, leading to the recruitment of DNA repair factors that repair and potentially modify the genome [9]. Discovery of off-target CRISPR-Cas genome editing in patient-derived cells and animal models is crucial for therapeutic applications, but exhibits low sensitivity due to reliance on detection of transient repair factor MRE11 binding events at Cas9-targeted sites. The disclosure is based on the discovery that inhibition of DNA-dependent protein kinase catalytic subunit (DNA-PKcs) accumulates MRE11 at Cas9-targeted sites, increasing the sensitivity of off-target detection up to over 5-fold in cell lines, induced pluripotent stem cells, and mice. Termed DISCOVER-Seq+, it is the most sensitive method to-date for discovery of off-target genome editing in vivo.

### DNA Repair

In cells, DNA is susceptible to many chemical alterations that can lead to mutations and there is a network of genes and their protein products involved in DNA repair mechanisms to correct damaged or inappropriate bases so that mutations do not accumulate.

DNA repair mechanisms include 1) direct chemical reversal of the damage and 2) excision repair. In excision repair, damaged base or bases are removed, then replaced/corrected in a localized area of DNA synthesis. Excision repair includes Base Excision Repair (BER), Nucleotide Excision Repair (NER) and Mismatch Repair (MMR), each of which uses specific sets of enzymes.

A large number of genes are reported to be involved in DNA repair mechanisms. These may be grouped broadly according to function e.g. Non-homologous end joining (NHEJ) genes, including XRCC4, LIG4, DNA-PK: Microhomology mediated end joining (MMEJ) genes, including MREII, XRCC1, LIG3; Homologous Recombination (HR) genes, including BRCA1, BRCA2, RAD51, LIG1; Mismatch repair (MMR) genes, including MLH1, MSH2, PMS2; Base Excision Repair (BER) genes, including Uracil DNA-glycosylase, AP-Endonuclease: Nucleotide Excision Repair (NER) genes, including XPC, XPD, XPA: DNA-cross-link repair genes, including FANCA, FANCB, FANCC; DNA-repair checkpoint genes, including ATM, ATR and p53. Other genes include those encoding DNA polymerases. There are two classes of polymerase that may be involved in DNA repair 1) those that readthrough errors, allowing them to remain and 2) those that proof-read. Among those that proof-read are polymerases that are involved in translesion synthesis, including DNA-pol ε and κ. DNA polymerases such as polymerase-δ contain proofreading activities and are primarily involved in replication error repair. When an error is detected, these polymerases halt the process of DNA replication, work backward to remove nucleotides from the daughter DNA chain until it is apparent that the improper nucleotide is gone, and then reinitiate the forward replication process. Mice with a point mutation in both copies of the Pold1 gene demonstrated a loss of proofreading activity by DNA polymerase-δ and developed epithelial cancers at a significantly higher rate than did mice with wild-type genes or with a single copy mutation (Robert E. Goldsby, et al. Proceedings of the National Academy of Sciences November 2002, 99 (24) 15560-15565: DOI: 10.1073/pnas.232340999).

O⁶-Methylguanine DNA methyltransferase (MGMT: DNA alkyltransferase) cleaves both methyl and ethyl adducts from guanine bases on the DNA structure. The reaction is not a catalytic (enzymatic) reaction but is stoichiometric (chemical), consuming one molecule of MGMT for each adduct removed. Cells that have been engineered to overexpress MGMT are more resistant to cancer, likely because they are able to negate a larger amount of alkylating damage. Niture, et al., reports an increase in MGMT expression by use of cysteine/glutathione enhancing drugs and natural antioxidants (Niture S K et al. Carcinogenesis. 28 (2): 378-389 (2007). DOI: 10.1093/carcin/bgl155).

A group of proteins known as mismatch excision repair (MMR) enzymes is capable of correcting errors of replication not detected by the proofreading activities of DNA polymerase. MMR enzymes excise an incorrect nucleotide from the daughter DNA and repair the strand using W-C pairing and the parent DNA strand as the correct template (Yang W. 2000. Structure and function of mismatch repair proteins. Mutation Research/DNA Repair. 460 (3-4): 245-256. DOI: 10.1016/s0921-8777 (00) 00030-6). This is especially crucial for errors generated during the replication of microsatellite regions, as the proofreading activity of DNA polymerase does not detect these errors. To a lesser degree, MMR enzymes also correct a variety of base pair anomalies resulting from DNA oxidation or alkylation. These mutations include modified base pairs containing O⁶-methylguanine and 8-oxoguanine, and carcinogen and cisplatin adducts (Iyer R R, et al., 2006. Chem. Rev. 106 (2): 302-323. DOI: 10.1021/cr0404794. Modrich P. 2006. J. Biol. Chem. 281 (41): 30305-30309. DOI: 10.1074/jbc.r600022200). Mutations in the human mismatch excision repair genes MSH2 and MLH1 are associated with hereditary non-polyposis colorectal cancer (HNPCC) syndrome (Müller A, Fishel R. 2002. Cancer Investigation. 20 (1): 102-109. DOI: 10.1081/cnv-120000371).

Base excision repair (BER) involves multiple enzymes to excise and replace a single damaged nucleotide base. The base modifications primarily repaired by BER enzymes are those damaged by endogenous oxidation and hydrolysis. A DNA glycosylase cleaves the bond between the nucleotide base and ribose, leaving the ribose phosphate chain of the DNA intact but resulting in an apurinic or apyrimidinic (AP) site. 8-Oxoguanine DNA glycosylase I(Ogg1) removes 7,8-dihydro-8-oxoguanine (8-oxoG), one of the base mutations generated by reactive oxygen species. Polymorphism in the human OGG1 gene is associated with the risk of various cancers such as lung and prostate cancer. Uracil DNA glycosylase, another BER enzyme, excises the uracil that is the product of cytosine deamination, thereby preventing the subsequent C→T point mutation (Lindahl T. 1974. Proceedings of the National Academy of Sciences. 71 (9): 3649-3653. DOI: 10.1073/pnas.71.9.3649). N-Methylpurine DNA glycosylase (MPG) is able to remove a variety of modified purine bases (Singer B, Hang B. 1997. Chem. Res. Toxicol. 10 (7): 713-732. DOI: 10.1021/tx970011e).

The AP sites in the DNA that result from the action of BER enzymes, as well as those that result from depyrimidination and depurination actions, are repaired by the action of AP-endonuclease 1 (APE1). APE1 cleaves the phosphodiester chain 5′ to the AP site. The DNA strand then contains a 3-hydroxyl group and a 5′-abasic deoxyribose phosphate. DNA polymerase β (Polβ) inserts the correct nucleotide based on the corresponding W-C pairing and removes the deoxyribose phosphate through its associated AP-lyase activity. The presence of X-ray repair cross-complementing group 1 (XRCC1) is necessary to form a heterodimer with DNA ligase III (LIG3). XRCC1 acts as a scaffold protein to present a non-reactive binding site for Polβ, and bring the Polβ and LIG3 enzymes together at the site of repair (Lindahl T. 1999. Quality Control by DNA Repair. 286 (5446): 1897-1905). Poly(ADP-ribose) polymerase (PARP-1) interacts with XRCC1 and Polβ and is a necessary component of the BER pathway. The final step in the repair is performed by LIG3, which connects the deoxyribose of the replacement nucleotide to the deoxyribosylphosphate backbone.

An alternative pathway called “long-patch BER” replaces a strand of nucleotides with a minimum length of 2 nucleotides. Repair lengths of 10 to 12 nucleotides have been reported. Longpatch BER requires the presence of proliferation cell nuclear antigen (PCNA), which acts as a scaffold protein for the restructuring enzymes.23 Other DNA polymerases, possibly Polo and Pole, are used to generate an oligonucleotide flap. The existing nucleotide sequence is removed by flap endonuclease-1 (FEN1). The oligonucleotide is then ligated to the DNA by DNA ligase I (LIG1), sealing the break and completing the repair.

Accordingly, any repair enzymes wherein modulation of function and/or activity leads to increase localization at the target nucleic acid sequence of a gene editing agent, are contemplated. In certain embodiments methods of detecting off-target genome editing comprise modulators of one or more DNA repair genes/proteins. Examples of genes (gene products) involved in DNA repair mechanisms include for example: ABL1, ALKBH1, ALKBH2, ALKBH3, APEX1, APEX2, APLF, APTX, ASF1A, ATF2, ATM, ATR, ATRIP, ATRX, ATXN3, BAZ1B, BLM, BRCA1, BRCA2, BRCC3, BRE, BRIP1, BTG2, C7ORF11, CCNH, CCNO, CDK7, CDKN2D, CETN2, CHAF1A, CHEK1, CHEK2, CIB1, CLK2, CNOT7, CSNK1D, CSNK1E, DCLRE1A, DCLRE1B, DCLRE1C, DDB1, DDB2, DDX11, DLGAP5, DMC1, DNA2, DNMT1, DUT, EME1, EME2, ERCC1, ERCC2, ERCC3, ERCC4, ERCC5, ERCC6, ERCC8, EYA1, EYA3, FAAP24, FAM175A, FAN1, FANCA, FANCB, FANCC, FANCD2, FANCE, FANCF, FANCG, FANCI, FANCL, FANCM, FEN1, FRAP1, GADD45A, GADD45G, GEN1, GIYD1, GTF2H1, GTF2H2, GTF2H3, GTF2H4, GTF2H5, H2AFX, HEL308, HMGB1, HMGB2, HUS1, IGHMBP2, IHPK3, KAT2A, KAT5, LIG1, LIG3, LIG4, MAD2L2, MBD4, MDC1, MEN1, MGMT, MIZF, MLH1, MLH3, MMS19, MNAT1, MPG, MRE11, MRE11A, MSH2, MSH3, MSH4, MSH5, MSH6, MUS81, MUTYH, NABP1, NABP2, NBN, NEIL1, NEIL2, NEIL3, NHEJ1, NPM1, NTHL1, NUDT1, OGG1, PALB2, PARG, PARP1, PARP2, PARP3, PCNA, PER1, PMS1, PMS2, PMS2L5, PNKP, POLA1, POLB, POLD1, POLE, POLE2, POLG, POLG2, POLH, POLI, POLK, POLL, POLM, POLN, POLQ, POLS, PRKCG, PRKDC, PRMT6, PRPF19. RAD1, RAD17, RAD18, RAD21, RAD23A, RAD23B, RAD50, RAD51, RAD51C, RAD51L1, RAD51L3, RAD52, RAD54B, RAD54L, RAD9A, RASSF7, RBBP8, RDM1, RECQL, RECQL4, RECQL5, REV1, REV3L, RNF168, RNF8, RPA1, RPA2, RPA3, RPA4, RPAIN, RPS27L, RRM2, RRM2B, RTEL1, RUVBL2, SETMAR, SETX, SHFM1, SIRT1, SMC1A, SMC3, SMC6, SMUG1, SOD1, SPO11, TADA3L, TCEA1, TDG, TDP1, TDP2, TNP1, TOP2A, TOPBP1, TP53, TP53BP1, TP73, TREX1, TREX2, TRIM28, TRIP13. TYMS, UBE2A, UBE2B, UBE2N, UBE2V1, UBE2V2, UIMC1, UNG, UPF1, USP1, UVRAG, VCP, WRN, XAB2, XPA, XPC, XRCC1, XRCC2, XRCC3, XRCC4, XRCC5, XRCC6, XRCC6BP1, YBX1.

Some examples of inhibitors of DNA repair gene products are as follows (Lodovichi S. Cervelli T. Pellicioli A. Galli A Inhibition of DNA Repair in Cancer Therapy: Toward a Multi-Target Approach. Int J Mol Sci. 2020: 21 (18): 6684. Published 2020 Sop 12. doi: 10.3390/ijms21186684): ATM: AZD0156, AZD1390, KU-55933, KU-60019, KU-59403. ATR: M6620, AZD6738, BAY1895344, VE-821. DNA-PK: M3814, VX-984, CC-115, MSC2490484A. CHKI: AZD7762, MK-8776. WEEI: AZD1775. MRE11: Mirin. RAD51: B02. RI-1, IBR120, CYT-0851. RAD52: F79, 6-OH-dopa, D-103, A5MP, AICAR/ZMP, NP-004255, F779-0434. TP53BP1: 153, UNC2170. XLF: G3. Pol-θ: Novobicin. BLM: ML216. DNA2: C5. WRN: NSC 19630, NSC617145. APE1: E3330. POL-β: NSC666715. ERCC1: NSC16168. FANCS: Phenylbutyrate. FANCD2: Curcumin, MLN4924. USP1/UAF1: ML323.

Modulators of DNA repair gene/gene products may be measured at number of different levels. In one embodiment, a functional assay may be performed to analyze e.g. the ability of a test compound to bind to a protein encoded by a DNA repair gene and/or the ability of that test compound to inhibit the function of that gene. Suitable assays for particular types of proteins involved in DNA repair will be familiar to those skilled in the art. For example, assays for non-homologous end joining (NHEJ), Microhomology mediated end joining (MMEJ), Homologous Recombination (HR), Mismatch repair (MMR), Base Excision Repair (BER), Nucleotide Excision Repair (NER), DNA-cross-link Repair, DNA-repair checkpoint and DNA polymerases are available.

### Gene Editing Agents

Compositions of the disclosure include at least one gene editing agent, comprising CRISPR-associated nucleases such as Cas9 and Cas12a gRNAs, Argonaute family of endonucleases, clustered regularly interspaced short palindromic repeat (CRISPR) nucleases, zinc-finger nucleases (ZFNs), transcription activator-like effector nucleases (TALENs), meganucleases, other endo- or exo-nucleases, or combinations thereof.

In certain embodiments, the compositions include isolated nucleic acid sequences encoding a Cpf1 (CRISPR from Prevotella and Francisella 1) endonuclease, and at least one guide RNA (gRNA), which is complementary to a target DNA sequence in the target gene. The gRNA directs the Cpf1 endonuclease to the target DNA sequence. The resulting double stranded breaks in the DNA inactivate the target gene by causing point mutations, insertions, deletions, or the complete excision of a stretch of DNA including the target gene.

In other embodiments, nuclease systems that can be used include, without limitation, zinc finger nucleases, transcription activator-like effector nucleases (TALENs), meganucleases, or any other system that can be used to degrade or interfere with viral nucleic acid without interfering with the regular function of the host's genetic material.

As referenced above, Argonaute is another potential gene editing system. Argonautes are a family of endonucleases that use 5′ phosphorylated short single-stranded nucleic acids as guides to cleave targets (Swarts, D. C. et al. The evolutionary journey of Argonaute proteins. Nat. Struct. Mol. Biol. 21, 743-753 (2014)). Similar to Cas9, Argonautes have key roles in gene expression repression and defense against foreign nucleic acids (Swarts, D. C. et al. Nat. Struct. Mol. Biol. 21, 743-753 (2014): Makarova, K. S., et al. Biol. Direct 4, 29 (2009). Molloy, S. Nat. Rev. Microbiol. 11, 743 (2013): Vogel, J. Science 344, 972-973 (2014). Swarts, D. C. et al. Nature 507, 258-261 (2014): Olovnikov, I., et al. Mol. Cell 51, 594-605 (2013)). However, Argonautes differ from Cas9 in many ways Swarts, D. C. et al. The evolutionary journey of Argonaute proteins. Nat. Struct. Mol. Biol. 21, 743-753 (2014)). Cas9 only exist in prokaryotes, whereas Argonautes are preserved through evolution and exist in virtually all organisms: although most Argonautes associate with single-stranded (ss) RNAs and have a central role in RNA silencing, some Argonautes bind ssDNAs and cleave target DNAs (Swarts, D. C. et al. Nature 507, 258-261 (2014): Swarts, D. C. et al. Nucleic Acids Res. 43, 5120-5129 (2015)). guide RNAs must have a 3′ RNA-RNA hybridization structure for correct Cas9 binding, whereas no specific consensus secondary structure of guides is required for Argonaute binding: whereas Cas9 can only cleave a target upstream of a PAM, there is no specific sequence on targets required for Argonaute. Once Argonaute and guides bind, they affect the physicochemical characteristics of each other and work as a whole with kinetic properties more typical of nucleic-acid-binding proteins (Salomon, W. E., et al. Cell 162, 84-95 (2015)).

CRISPR (Clustered Regularly Interspaced Short Palindromic Repeats) is found in bacteria and is believed to protect the bacteria from phage infection. It has recently been used as a means to alter gene expression in eukaryotic DNA, but has not been proposed as an anti-viral therapy or more broadly as a way to disrupt genomic material. Rather, it has been used to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327: Bhaya et al., Annu Rev Genet (2011) 45:273-297: Wiedenheft et al., Nature (2012) 482:331-338): Jinek M et al., Science (2012) 337:816-821: Cong L et al., Science (2013) 339:819-823: Jinek M et al., (2013) eLife 2: e00471: Mali P et al. (2013) Science 339:823-826: Qi L S et al. (2013) Cell 152:1173-1183: Gilbert L A et al. (2013) Cell 154:442-451: Yang H et al. (2013) Cell 154:1370-1379; and Wang H et al. (2013) Cell 153:910-918).

CRISPR methodologies employ a nuclease, CRISPR-associated (Cas), that complexes with small RNAs as guides (gRNAs) to cleave DNA in a sequence-specific manner upstream of the protospacer adjacent motif (PAM) in any genomic location. CRISPR may use separate guide RNAs known as the crRNA and tracrRNA. These two separate RNAs have been combined into a single RNA to enable site-specific mammalian genome cutting through the design of a short guide RNA. Cas and guide RNA (gRNA) may be synthesized by known methods. Cas/guide-RNA (gRNA) uses a non-specific DNA cleavage protein Cas, and an RNA oligonucleotide to hybridize to target and recruit the Cas/gRNA complex. See Chang et al., 2013, Cell Res. 23:465-472; Hwang et al., 2013, Nat. Biotechnol. 31:227-229; Xiao et al., 2013, Nucl. Acids Res. 1-11.

In general, the CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNase domains, protein-protein interaction domains, dimerization domains, as well as other domains. The mechanism through which CRISPR/Cas9-induced mutations inactivate the provirus can vary. For example, the mutation can affect proviral replication, and viral gene expression. The mutation can comprise one or more deletions. The size of the deletion can vary from a single nucleotide base pair to about 10,000 base pairs. In some embodiments, the deletion can include all or substantially all of the proviral sequence. In some embodiments the deletion can eradicate the provirus. The mutation can also comprise one or more insertions, that is, the addition of one or more nucleotide base pairs to the proviral sequence. The size of the inserted sequence also may vary, for example from about one base pair to about 300 nucleotide base pairs. The mutation can comprise one or more point mutations, that is, the replacement of a single nucleotide with another nucleotide. Useful point mutations are those that have functional consequences, for example, mutations that result in the conversion of an amino acid codon into a termination codon, or that result in the production of a nonfunctional protein.

The RNA-guided Cas9 biotechnology induces genome editing without detectable off-target effects. This technique takes advantage of the genome defense mechanisms in bacteria that CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). Cas9 belongs to the type II CRISPR/Cas system and has strong endonuclease activity to cut target DNA.

In certain embodiments, the CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In one embodiment, the RNA-guided endonuclease is derived from a type II CRISPR/Cas system. The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called spacer) and a trans-activated small RNA (tracrRNA) that serves as a guide for ribonuclease III-aided processing of pre-crRNA. The crRNA: tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM). The crRNA and tracrRNA can be expressed separately or engineered into an artificial fusion small guide RNA (sgRNA) via a synthetic stem loop (AGAAAU) to mimic the natural crRNA/tracrRNA duplex. Such sgRNA, like shRNA, can be synthesized or in vitro transcribed for direct RNA transfection or expressed from U6 or H1-promoted RNA expression vector, although cleavage efficiencies of the artificial sgRNA are lower than those for systems with the crRNA and tracrRNA expressed separately. Therefore, the Cas9 gRNA technology requires the expression of the Cas9 protein and gRNA, which then form a gene editing complex at the specific target DNA binding site within the target genome and inflict cleavage/mutation of the target DNA.

However, the present disclosure is not limited to the use of Cas9-mediated gene editing. Rather, the present disclosure encompasses the use of other CRISPR-associated peptides, which can be targeted to a targeted sequence using a gRNA and can edit to target site of interest. For example, in some embodiments, the disclosure utilizes Cas12a (also known as Cpf1) to edit the target site of interest.

CRISPR-Cas systems include Type I CRISPR-Cas system, Type II CRISPR-Cas system, Type III CRISPR-Cas system, and derivatives thereof. CRISPR-Cas systems include engineered and/or programmed nuclease systems derived from naturally accruing CRISPR-Cas systems. In certain embodiments, CRISPR-Cas systems contain engineered and/or mutated Cas proteins. In some embodiments, nucleases generally refer to enzymes capable of cleaving the phosphodiester bonds between the nucleotide subunits of nucleic acids. In some embodiments, endonucleases are generally capable of cleaving the phosphodiester bond within a polynucleotide chain. Nickases refer to endonucleases that cleave only a single strand of a DNA duplex.

In some embodiments, the CRISPR/Cas system used herein can be a type I, a type II, or a type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, CasX, CasΦ, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. By way of further example, in some embodiments, the CRISPR-Cas protein is a Cas1, Cas1B, Cas2, Cas3, Cas4, Cas5, Cash, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx16, CsaX, Csx3, Csx1, Csx15, Csf1, Csf2, Csf3, Csf4, Cas9, Cas12 (e.g., Cas12a, Cas12b, Cas12c, Cas12d, Cas12k, Cas12j/CasΦ, Cas12L etc.), Cas13 (e.g., Cas13a, Cas13b (such as Cas13b-t1, Cas13b-t2, Cas13b-t3), Cas13c, Cas13d, etc.), Cas14, CasX, CasY, or an engineered form of the Cas protein. In some embodiments, the CRISPR/Cas protein or endonuclease is Cas9. In some embodiments, the CRISPR/Cas protein or endonuclease is Cas12. In certain embodiments, the Cas 12 polypeptide is Cas12a, Cas12b, Cas12c, Cas12d, Cas12e, Cas12g, Cas12h, Cas12i, Cas12L or Cas 12J. In some embodiments, the CRISPR/Cas protein or endonuclease is CasX. In some embodiments, the CRISPR/Cas protein or endonuclease is CasY. In some embodiments, the CRISPR/Cas protein or endonuclease is CasΦ.

In some embodiments, the Cas9 protein can be from or derived from: Staphylococcus aureus, Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Fine goldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In some embodiments, the composition comprises a CRISPR-associated (Cas) protein, or functional fragment or derivative thereof. In some embodiments, the Cas protein is an endonuclease, including but not limited to the Cas9 nuclease. In some embodiments, the Cas9 protein comprises an amino acid sequence identical to the wild type Streptococcus pyogenes or Staphylococcus aureus Cas9 amino acid sequence. In some embodiments, the Cas protein comprises the amino acid sequence of a Cas protein from other species, for example other Streptococcus species, such as thermophilus: Pseudomonas aeruginosa. Escherichia coli, or other sequenced bacteria genomes and archaea, or other prokaryotic microorganisms. Other Cas proteins, useful for the present disclosure, known or can be identified, using methods known in the art (see e.g., Esvelt et al., 2013, Nature Methods, 10:1116-1121). In some embodiments, the Cas protein comprises a modified amino acid sequence, as compared to its natural source.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the Cas protein. The CRISPR/Cas-like protein can also be truncated or modified to optimize the activity of the effector domain of the Cas protein.

The disclosed CRISPR-Cas compositions should also be construed to include any form of a protein having substantial homology to a Cas protein (e.g., Cas9, saCas9, Cas9 protein) disclosed herein. In some embodiments, a protein which is “substantially homologous” is about 50% homologous, about 70% homologous, about 80% homologous, about 90% homologous, about 95% homologous, or about 99% homologous to amino acid sequence of a Cas protein disclosed herein. The Cas9 can be an orthologous. Six smaller Cas9 orthologues have been used and reports have shown that Cas9 from Staphylococcus aureus (SaCas9) can edit the genome with efficiencies similar to those of SpCas9, while being more than 1 kilobase shorter.

Other Cas peptides, useful for the present disclosure, known or can be identified, using methods known in the art (see e.g., Esvelt et al., 2013, Nature Methods, 10:1116-1121). In certain embodiments, the Cas peptide may comprise a modified amino acid sequence, as compared to its natural source. For example, in some embodiments, the wild type Streptococcus pyogenes Cas9 sequence can be modified. In certain embodiments, the amino acid sequence can be codon optimized for efficient expression in human cells (i.e., “humanized) or in a species of interest. A humanized Cas9 nuclease sequence can be for example, the Cas9 nuclease sequence encoded by any of the expression vectors listed in Genbank accession numbers KM099231.1 GL669193757: KM099232.1 GL669193761: or KM099233.1 GL669193765. Alternatively, the Cas9 nuclease sequence can be for example, the sequence contained within a commercially available vector such as PX330 or PX260 from Addgene (Cambridge, MA). In some embodiments, the Cas9 endonuclease can have an amino acid sequence that is a variant or a fragment of any of the Cas9 endonuclease sequences of Genbank accession numbers KM099231.1 GL669193757: KM099232.1 GL669193761: or KM099233.1 GL669193765 or Cas9 amino acid sequence of PX330 or PX260 (Addgene, Cambridge, MA).

The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution).

In certain embodiments, the Cas peptide is a mutant Cas9, wherein the mutant Cas9 reduces the off-target effects, as compared to wild-type Cas9. In some embodiments, the mutant Cas9 is a Streptococcus pyogenes Cas9 (SpCas9) variant. In some embodiments, SpCas9 variants comprise one or more point mutations, including, but not limited to R780A, K810A, K848A, K855A, H982A, K1003A, and R1060A (Slaymaker et al., 2016, Science, 351 (6268): 84-88). In some embodiments, SpCas9 variants comprise D1135E point mutation (Kleinstiver et al., 2015, Nature, 523 (7561): 481-485). In some embodiments, SpCas9 variants comprise one or more point mutations, including, but not limited to N497A, R661A, Q695A, Q926A, D1135E, L169A, and Y450A (Kleinstiver et al., 2016, Nature, doi: 10.1038/nature 16526). In some embodiments, SpCas9 variants comprise one or more point mutations, including but not limited to M495A, M694A, and M698A. Y450 is involved with hydrophobic base pair stacking. N497, R661, Q695, Q926 are involved with residue to base hydrogen bonding contributing to off-target effects. N497 hydrogen bonding through peptide backbone. L169A is involved with hydrophobic base pair stacking. M495A, M694A, and H698A are involved with hydrophobic base pair stacking.

In some embodiments, SpCas9 variants comprise one or more point mutations at one or more of the following residues: R780, K810, K848, K855, H982, K1003, R1060, D1135, N497, R661, Q695, Q926, L169, Y450, M495, M694, and M698. In some embodiments, SpCas9 variants comprise one or more point mutations selected from the group of: R780A, K810A, K848A, K855A, H982A, K1003A, R1060A, D1135E, N497A, R661A, Q695A, Q926A, L169A, Y450A, M495A, M694A, and M698A.

In some embodiments, the SpCas9 variant comprises the point mutations, relative to wildtype SpCas9, of N497A, R661A, Q695A, and Q926A. In some embodiments, the SpCas9 variant comprises the point mutations, relative to wildtype SpCas9, of N497A, R661A, Q695A, Q926A, and D1135E. In some embodiments, the SpCas9 variant comprises the point mutations, relative to wildtype SpCas9, of N497A, R661A, Q695A, Q926A, and L169A. In some embodiments, the SpCas9 variant comprises the point mutations, relative to wildtype SpCas9, of N497A, R661A, Q695A, Q926A, and Y450A. In some embodiments, the SpCas9 variant comprises the point mutations, relative to wildtype SpCas9, of N497A, R661A, Q695A, Q926A, and M495A. In some embodiments, the SpCas9 variant comprises the point mutations, relative to wildtype SpCas9, of N497A, R661A, Q695A, Q926A, and M694A. In some embodiments, the SpCas9 variant comprises the point mutations, relative to wildtype SpCas9, of N497A, R661A, Q695A, Q926A, and H698A. In some embodiments, the SpCas9 variant comprises the point mutations, relative to wildtype SpCas9, of N497A, R661A, Q695A, Q926A, D1135E, and L169A. In some embodiments, the SpCas9 variant comprises the point mutations, relative to wildtype SpCas9, of N497A, R661A, Q695A, Q926A, D1135E, and Y450A. In some embodiments, the SpCas9 variant comprises the point mutations, relative to wildtype SpCas9, of N497A, R661A, Q695A, Q926A, D1135E, and M495A. In some embodiments, the SpCas9 variant comprises the point mutations, relative to wildtype SpCas9, of N497A, R661A, Q695A, Q926A, D1135E, and M694A. In some embodiments, the SpCas9 variant comprises the point mutations, relative to wildtype SpCas9, of N497A, R661A, Q695A, Q926A, D1135E, and M698A.

In some embodiments, the mutant Cas9 comprises one or more mutations that alter PAM specificity (Kleinstiver et al., 2015, Nature, 523 (7561): 481-485; Kleinstiver et al., 2015, Nat Biotechnol, 33 (12): 1293-1298). In some embodiments, the mutant Cas9 comprises one or more mutations that alter the catalytic activity of Cas9, including but not limited to D10A in RuvC and H840A in HNH (Cong et al., 2013: Science 339:919-823, Gasiubas et al., 2012: PNAS 109: E2579-2586 Jinek et al., 2012; Science 337:816-821).

In addition to the wild type and variant Cas9 endonucleases described, embodiments of the disclosure also encompass CRISPR systems including newly developed “enhanced-specificity” S. pyogenes Cas9 variants (eSpCas9), which dramatically reduce off target cleavage. These variants are engineered with alanine substitutions to neutralize positively charged sites in a groove that interacts with the non-target strand of DNA. This aim of this modification is to reduce interaction of Cas9 with the non-target strand, thereby encouraging re-hybridization between target and non-target strands. The effect of this modification is a requirement for more stringent Watson-Crick pairing between the gRNA and the target DNA strand, which limits off-target cleavage (Slaymaker, I. M. et al. (2015) DOI: 10.1126/science.aad5227).

In certain embodiments, three variants found to have the best cleavage efficiency and fewest off-target effects: SpCas9 (K855A), SpCas9 (K810A/K1003A/R1060A) (a.k.a. eSpCas9 1.0), and SpCas9 (K848A/K1003A/R1060A) (a.k.a. eSPCas9 1.1) are employed in the compositions. The disclosure is by no means limited to these variants, and also encompasses all Cas9 variants (Slaymaker, I. M. et al. (2015)). The present disclosure also includes another type of enhanced specificity Cas9 variant, “high fidelity” spCas9 variants (HF-Cas9). Examples of high fidelity variants include SpCas9-HF1 (N497A/R661A/Q695A/Q926A), SpCas9-HF2 (N497A/R661A/Q695A/Q926A/D1135E), SpCas9-HF3 (N497A/R661A/Q695A/Q926A/L169A), SpCas9-HF4 (N497A/R661A/Q695A/Q926A/Y450A). Also included are all SpCas9 variants bearing all possible single, double, triple and quadruple combinations of N497A, R661A, Q695A, Q926A or any other substitutions (Kleinstiver, B. P. et al., 2016, Nature. DOI: 10.1038/nature16526).

Accordingly, in certain embodiments, a Cas9 variant comprises a human-optimized Cas9: a nickase mutant Cas9: saCas9: enhanced-fidelity SaCas9 (efSaCas9): SpCas9 (K855a): SpCas9 (K810A/K1003A/r1060A): SpCas9 (K848A/K1003A/R1060A); SpCas9 N497A, R661A, Q695A, Q926A: SpCas9 N497A, R661A, Q695A, Q926A, D1135E: SpCas9 N497A, R661A, Q695A, Q926A L169A: SpCas9 N497A, R661A, Q695A, Q926A Y450A: SpCas9 N497A, R661A, Q695A, Q926A M495A: SpCas9 N497A, R661A, Q695A, Q926A M694A: SpCas9 N497A, R661A, Q695A, Q926A H698A: SpCas9 N497A, R661A, Q695A, Q926A, D1135E, L169A: SpCas9 N497A, R661A, Q695A, Q926A, D1135E, Y450A: SpCas9 N497A, R661A, Q695A, Q926A, D1135E, M495A: SpCas9 N497A, R661A, Q695A, Q926A, D1135E, M694A: SpCas9 N497A, R661A, Q695A, Q926A, D1135E, M698A: SpCas9 R661A, Q695A, Q926A: SpCas9 R661A, Q695A, Q926A, D1135E: SpCas9 R661A, Q695A, Q926A, L169A: SpCas9 R661A, Q695A, Q926A Y450A: SpCas9 R661A, Q695A, Q926A M495A: SpCas9 R661A, Q695A, Q926A M694A; SpCas9 R661A, Q695A, Q926A H698A: SpCas9 R661A, Q695A, Q926A D1135E L169A; SpCas9 R661A, Q695A, Q926A D1135E Y450A: SpCas9 R661A, Q695A, Q926A D1135E M495A: or SpCas9 R661A, Q695A, Q926A, D1135E or M694A.

### Delivery Vehicles

For discovery of gene-editing, e.g. CRISPR, off-target editing in primary cells and in vivo, vectors are provided for expression of the gene editing agents and gRNAs. In certain embodiments, synthetic nanoparticles for co-delivery of one or more nucleic acids, proteins, DNA repair modulators are provided. Delivery vehicles include polymeric nanoparticles, micelles, lipid micelles, liposomes, and hybrid lipid-polymer nanoparticles.

Nucleic Acids and Vectors: In some embodiments, the composition of the disclosure comprises an isolated nucleic acid encoding one or more elements of the CRISPR-Cas system described herein. For example, in some embodiments, the composition comprises an isolated nucleic acid encoding at least one guide nucleic acid (e.g., gRNA). In some embodiments, the composition comprises an isolated nucleic acid encoding a Cas peptide, or functional fragment or derivative thereof. In some embodiments, the composition comprises an isolated nucleic acid encoding at least one guide nucleic acid (e.g., gRNA) and encoding a Cas peptide, or functional fragment or derivative thereof. In some embodiments, the composition comprises an isolated nucleic acid encoding at least one guide nucleic acid (e.g., gRNA) and further comprises an isolated nucleic acid encoding a Cas peptide, or functional fragment or derivative thereof.

In some embodiments, the composition comprises at least one isolated nucleic acid encoding a gRNA, where the gRNA is substantially complementary to a target sequence. In some embodiments, the composition comprises at least one isolated nucleic acid encoding a gRNA, where the gRNA is complementary to a target sequence having at least 75%, 80%, 85%, 90%, 95%, 96%, 97%, 98%, or 99% sequence homology to a target sequence.

In some embodiments, the composition comprises at least one isolated nucleic acid encoding a Cas peptide described elsewhere herein, or a functional fragment or derivative thereof. In some embodiments, the composition comprises at least one isolated nucleic acid encoding a Cas peptide having at least 75%, 80%, 85%, 90%, 95%, 96%, 97%, 98%, or 99% amino acid sequence homology with a Cas peptide described elsewhere herein.

The isolated nucleic acid may comprise any type of nucleic acid, including, but not limited to DNA and RNA. For example, in some embodiments, the composition comprises an isolated DNA, including for example, an isolated cDNA, encoding a gRNA or peptide of the disclosure, or functional fragment thereof. In some embodiments, the composition comprises an isolated RNA encoding a peptide of the disclosure, or a functional fragment thereof. The isolated nucleic acids may be synthesized using any method known in the art.

The present disclosure can comprise use of a vector in which the isolated nucleic acid described herein is inserted. The art is replete with suitable vectors that are useful in the present disclosure. Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding): components that influence uptake of the vector nucleic acid by the cell: components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques. 34:167-171 (2003). A large variety of such vectors is known in the art and is generally available.

In brief summary, the expression of natural or synthetic nucleic acids encoding an RNA and/or peptide is typically achieved by operably linking a nucleic acid encoding the RNA and/or peptide or portions thereof to a promoter, and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present disclosure may also be used for nucleic acid immunization and gene therapy, using standard gene delivery protocols. Methods for gene delivery are known in the art. See. e.g., U.S. Pat. Nos. 5,399,346, 5,580,859, 5,589,466, incorporated by reference herein in their entireties. In another embodiment, the disclosure provides a gene therapy vector.

The isolated nucleic acid of the disclosure can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584: WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In some embodiments, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce post-mitotic cells in a stable and efficient manner. Expression of a particular gene contained within an AAV vector can be specifically targeted to one or more types of cells by choosing the appropriate combination of AAV serotype, promoter, and delivery method.

Further provided are nucleic acids encoding the CRISPR-Cas systems described herein. Provided herein are adeno-associated virus (AAV) vectors comprising nucleic acids encoding the CRISPR-Cas systems described herein. In certain instances, an AAV vector includes to any vector that comprises or derives from components of AAV and is suitable to infect mammalian cells, including human cells, of any of a number of tissue types, such as brain, heart, lung, skeletal muscle, liver, kidney, spleen, or pancreas, whether in vitro or in vivo. In certain instances, an AAV vector includes an AAV type viral particle (or virion) comprising a nucleic acid encoding a protein of interest (e.g. CRISPR-Cas systems described herein). In some embodiments, as further described herein, the AAVs disclosed herein are be derived from various serotypes, including combinations of serotypes (e.g., “pseudotyped” AAV) or from various genomes (e.g., single-stranded or self-complementary). In some embodiments, the AAV vector is a human serotype AAV vector. In such embodiments, a human serotype AAV is derived from any known serotype, e.g., from AAV1, AAV2, AAV4, AAV6, or AAV9. In some embodiments, the serotype is AAV1, AAV2, AAV3, AAV4, AAV5, AAV6, AAV7, AAV8, AAV9, AAV10, AAV11, AAVDJ, or AAVDJ/8.

In some embodiments, the composition includes a vector derived from an adeno-associated virus (AAV). AAV vectors possess a number of features that render them ideally suited for gene therapy, including a lack of pathogenicity, minimal immunogenicity, and the ability to transduce post-mitotic cells in a stable and efficient manner. Expression of a particular gene contained within an AAV vector can be specifically targeted to one or more types of cells by choosing the appropriate combination of AAV serotype, promoter, and delivery method.

A variety of different AAV capsids have been described and can be used, although AAV which preferentially target the liver and/or deliver genes with high efficiency are particularly desired. The sequences of the AAV8 are available from a variety of databases. While the examples utilize AAV vectors having the same capsid, the capsid of the gene editing vector and the AAV targeting vector are the same AAV capsid. Another suitable AAV is, e.g., rh10 (WO 2003/042397). Still other AAV sources include, e.g., AAV9 (see, for example, U.S. Pat. No. 7,906,111; US 2011-0236353-A1), and/or hu37 (see, e.g., U.S. Pat. No. 7,906,111; US 2011-0236353-A1), AAV1, AAV2, AAV3, AAV4, AAV5, AAV6, AAV6.2, AAV7, AAV8, (U.S. Pat. Nos. 7,790,449; 7,282,199, WO 2003/042397; WO 2005/033321, WO 2006/110689; U.S. Pat. Nos. 7,790,449; 7,282,199; 7,588,772). Still other AAV can be selected, optionally taking into consideration tissue preferences of the selected AAV capsid.

In some embodiments, AAV vectors disclosed herein include a nucleic acid encoding a CRISPR-Cas systems described herein. In some embodiments, the nucleic acid also includes one or more regulatory sequences allowing expression and, in some embodiments, secretion of the protein of interest, such as e.g., a promoter, enhancer, polyadenylation signal, an internal ribosome entry site (“IRES”), a sequence encoding a protein transduction domain (“PTD”), and the like. Thus, in some embodiments, the nucleic acid comprises a promoter region operably linked to the coding sequence to cause or improve expression of the protein of interest in infected cells. Such a promoter can be ubiquitous, cell- or tissue-specific, strong, weak, regulated, chimeric, etc., for example, to allow efficient and stable production of the protein in the infected tissue. In certain embodiments, the promoter is homologous to the encoded protein, or heterologous, although generally promoters of use in the disclosed methods are functional in human cells. Examples of regulated promoters include, without limitation, Tet on/off element-containing promoters, rapamycin-inducible promoters, tamoxifen-inducible promoters, and metallothionein promoters. In certain embodiments. other promoters used include promoters that are tissue specific for tissues such as kidney, spleen, and pancreas. Examples of ubiquitous promoters include viral promoters, particularly the CMV promoter, the RSV promoter, the SV40 promoter, etc., and cellular promoters such as the phosphoglycerate kinase (PGK) promoter and the b-actin promoter.

In some embodiments, the recombinant AAV vector comprises packaged within an AAV capsid, a nucleic acid, generally containing a 5′ AAV ITR, the expression cassettes described herein and a 3′ AAV ITR. As described herein, in some embodiments, an expression cassette contains regulatory elements for an open reading frame(s) within each expression cassette and the nucleic acid optionally contains additional regulatory elements. The AAV vector, in some embodiments, comprises a full-length AAV 5′ inverted terminal repeat (ITR) and a full-length 3′ ITR. A shortened version of the 5′ ITR, termed ΔITR, has been described in which the D-sequence and terminal resolution site (trs) are deleted. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription (see, for example, D M McCarty et al., “Self-complementary recombinant adeno-associated virus (scAAV) vectors promote efficient transduction independently of DNA synthesis”, Gene Therapy, (August 2001): see also, for example, U.S. Pat. Nos. 6,596,535; 7,125,717; and 7,456,683). Where a pseudotyped AAV is to be produced, the ITRs are selected from a source which differs from the AAV source of the capsid. For example, in some embodiments, AAV2 ITRs are selected for use with an AAV capsid having a particular efficiency for a selected cellular receptor, target tissue or viral target. In some embodiments, the ITR sequences from AAV2, or the deleted version thereof (AITR), are used for convenience and to accelerate regulatory approval (i.e. pseudotyped). In some embodiments, a single-stranded AAV viral vector is used.

Methods for generating and isolating AAV viral vectors suitable for delivery to a subject are known in the art (see, for example, U.S. Pat. No. 7,790,449: U.S. Pat. No. 7,282,199: WO 2003/042397: WO 2005/033321, WO 2006/110689; and U.S. Pat. No. 7,588,772 B2, U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065). In one system, a producer cell line is transiently transfected with a construct that encodes the transgene flanked by ITRs and a construct(s) that encodes rep and cap. In a second system, a packaging cell line that stably supplies rep and cap is transfected (transiently or stably) with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus UL5, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors.

The CRISPR-Cas systems, for instance a Cas9, and/or any of the present RNAs, for instance a guide RNA, can be delivered using adeno associated virus (AAV), lentivirus, adenovirus or other viral vector types, or combinations thereof. Cas9 and one or more guide RNAs can be packaged into one or more viral vectors. In some embodiments, the viral vector is delivered to the tissue of interest by, for example, an intramuscular injection, while other times the viral delivery is via intravenous, transdermal, intranasal, oral, mucosal, or other delivery methods. Such delivery can be either via a single dose, or multiple doses. One skilled in the art understands that the actual dosage to be delivered herein can vary greatly depending upon a variety of factors, such as the vector chose, the target cell, organism, or tissue, the general condition of the subject to be treated, the degree of transformation/modification sought, the administration route, the administration mode, the type of transformation/modification sought, etc.

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some embodiments. The adenovirus vector results in a shorter term expression (e.g., less than about a month) than adeno-associated virus, in some embodiments, may exhibit much longer expression. The particular vector chosen will depend upon the target cell and the condition being treated.

In certain embodiments, the vector also includes conventional control elements which are operably linked to the transgene in a manner which permits its transcription, translation and/or expression in a cell transfected with the plasmid vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

The selection of appropriate promoters can readily be accomplished. In certain aspects, one would use a high expression promoter. One example of a suitable promoter is the immediate early cytomegalovirus (CMV) promoter sequence. This promoter sequence is a strong constitutive promoter sequence capable of driving high levels of expression of any polynucleotide sequence operatively linked thereto. The Rous sarcoma virus (RSV) and MMT promoters may also be used. Certain proteins can be expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication.

Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatinine kinase promoter. Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Enhancer sequences found on a vector also regulates expression of the gene contained therein. Typically, enhancers are bound with protein factors to enhance the transcription of a gene. Enhancers may be located upstream or downstream of the gene it regulates. Enhancers may also be tissue-specific to enhance transcription in a specific cell or tissue type. In some embodiments, the vector of the present disclosure comprises one or more enhancers to boost transcription of the gene present within the vector.

In order to assess the expression of the nucleic acid and/or peptide, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Polymeric Particles: Nanoparticles can be formed of biodegradable, biocompatible polymers for co-delivery of the nucleic acids. Typically the nanoparticles are formed of one or more hydrophobic polymers, optionally including amphiphilic polymers in the form of a blend where the hydrophilic polymers orient to the exterior of the nanoparticle, and/or hydrophilic polymers on the surface to avoid uptake by the reticuloendothelial system and enhance phagocytosis. Cationic polymers may be utilized to increase encapsulation of nucleic acids.

Hydrophobic cationic material, hydrophobic polymer and/or the hydrophobic portion of amphiphilic materials provide a non-polar polymer matrix for loading non-polar drugs, protect and promoting siRNA molecule retention inside the NP core, and control drug release. The hydrophilic portion of the amphiphilic material can form a corona around the particle which prolongs circulation of the particles in the blood stream and decreases uptake by the RES. In one embodiment, the amphiphilic material is a hydrophobic, biodegradable polymer terminated with a hydrophilic block.

Biocompatible polymers include, but are not limited to, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polylactides, polyglycolides, polysiloxanes, polyurethanes and copolymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt: polyacrylic acid polymers such as polymers of acrylic and methacrylic esters such as poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyalkylenes such as polyethylene, polypropylene poly(ethylene glycol), poly(ethylene oxide), and poly(ethylene terephthalate), poly(vinyl alcohols), poly(vinyl acetate), poly vinyl chloride polystyrene and polyvinylpryrrolidone, derivatives thereof, linear and branched copolymers and block copolymers thereof, and blends thereof.

Exemplary biodegradable polymers include, but are not limited to, polyesters, poly(ortho esters), poly(ethylene imines), poly(caprolactones), poly(hydroxybutyrates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. In particularly preferred embodiments the polymeric core contains biodegradable hydrophobic polyesters such as poly(lactic acid), poly(glycolic acid), and poly(lactic-co-glycolic acid), and/or these polymers conjugated to polyalkylene oxides such as polyethylene glycol or block copolymers such as the polypropylene oxide-polyethylene oxide PLURONICS™.

The molecular weight of the biodegradable oligomeric or polymeric segment or polymer can be varied to tailor the properties of the polymer. Exemplary molecular weights include between about 150 Da and about 100 kDa, more preferably between about 1 kDa and about 75 kDa, most preferably between about 5 kDa and about 50 kDa.

In some embodiments, the hydrophilic polymers or segment(s) or block(s) include, but are not limited to, homo polymers or copolymers of polyalkene glycols, such as poly(ethylene glycol), poly(propylene glycol), poly(butylene glycol), and acrylates and acrylamides, such as hydroxyethyl methacrylate and hydroxypropyl-methacrylamide. The hydrophilic polymer segment typically has a molecular weight of between about 150 Da and about 20 kDa, more preferably between about 500 Da and about 10 kDa, most preferably between about 1 kDa and about 5 kDa.

The nanoparticles can be formed of a mixture or blend of polymers. In preferred embodiments, these are a blend of amphiphilic polymers, preferably copolymers of modified polyethylene glycol (PEG) and polyesters, such as various forms of PLGA-PEG or PLA-PEG copolymers, collectively referred to herein as “PEGylated polymers”, some hydrophobic polymer such as PLGA, PLA or PGA, and/or some may be hydrophilic polymer such as a PEG or PEG derivative. Some may be modified by conjugation to a targeting agent, a cell adhesion or a cell penetrating peptide.

In some embodiments, the cationic material is a material that is cationic at the time the hydrophobic cationic material is prepared or becomes cationic under physiological conditions. In some embodiments, the cationic material contains one or more amine containing moieties, such as amine containing small molecules, amine-containing polymers, such as PEI, and amine-containing macromolecules, such as dendrimers (see the structures below). The cationic moieties are functionalized with one or more hydrophobic/lipid moieties, such as lipophilic alkyl chains (e.g., C₆-C₃₀, C₆-C₂₄), cholesterol, saturated or unsaturated fatty acids, etc.

Stimuli responsive polymers are well known in the art. Stimuli responsive amphiphilic polymers are responsive to a stimulus such as a pH change, redox change, temperature change, exposure to light or other stimuli, including binding to a target. Stimuli responsive polymers are reviewed by James, et al., Acta Pharma. Sinica B 4 (2): 120-127 (2014). The following is a list of exemplary polymers categorized by responsive to various stimuli: Temperature: POLOXAMERS™, poly(N-alkylacrylamide) s, poly(N-vinylcaprolactam) s, cellulose, xyoglucan, and chitosan: pH: poly(methacrylic acid) s, poly(vinylpyridine) s, and poly(vinylimmidazole) s: light: modified poly(acrylamide) s: electric field: sulfonated polystyrenes, poly(thiophene) s, and poly(ethyloxazoline) s; ultrasound: ethylenevinylacetate.

Exemplary pH dependent polymers include dendrimers formed of poly(lysine), poly(hydroxyproline), PEG-PLA, Poly(propyl acrylic acid), Poly(ethacrylic acid), CARBOPOLL™, Polysilamine, EUDRAGIT™ S-100 EUDRAGIT® L-100, Chitosan, PMAA-PEG copolymer, sodium alginate (Ca²⁺). The ionic pH sensitive polymers are able to accept or release protons in response to pH changes. These polymers contain acid groups (carboxylic or sulfonic) or basic groups (ammonium salts) so that the pH sensitive polymers are polyelectrolytes that have in their structure acid or basic groups that can accept or release protons in response to pH changes in the surrounding environment. pH values from several tissues and cell compartments can be used to trigger release in these tissues. For example, the pH of blood is 7.4-7.5; stomach is 1.0-3.0; duodenum is 4.8-8.2; colon is 7.0-7.5: lysosome is 4.5-5.0; Golgi complex is 6.4; tumor—extracellular medium is 6.2-7.2. pH is typically lower in areas of infection or inflammation. Examples of thermosensitive polymers include the poly(N-substituted acrylamide) polymers such as poly(N-isopoprylacrilamide) (PNIPAAm), poly(N,N′-diethyl acrylamide), poly(dimethylamino ethyl methacrylate and poly(N-(L)-(1-hydroxymethyl) propyl methacrylamide).

Biologically responsive polymer systems are increasingly important in various biomedical applications. The major advantage of bioresponsive polymers is that they can respond to the stimuli that are inherently present in the natural system. Bioresponsive polymeric systems mainly arise from common functional groups that are known to interact with biologically relevant species, and in other instances the synthetic polymer is conjugated to a biological component. Bioresponsive polymers include antigen-responsive polymers, glucose-sensitive polymers, and enzyme-responsive polymers.

Lipid-Based Delivery Vehicles: Nanoparticles may include one or more lipids, may be in the form of a liposome, may include a lipid monolayer or bilayer, or be formed of micelles. In some embodiments, nanoparticles include a polymeric core surrounded by a lipid layer (e.g., lipid bilayer, lipid monolayer, etc.). In some embodiments, a nanoparticle includes a non-polymeric core (e.g., metal particle, quantum dot, ceramic particle, bone particle, etc.) surrounded by a lipid layer (e.g., lipid bilayer, lipid monolayer, etc.).

The percent of lipid in the nanoparticles can be from greater than 0% to 99% by weight, inclusive, from 10% to 99% by weight, from 25% to 99% by weight, from 50% to 99% by weight, or from 75% to 99% by weight. In some embodiments, the percent of lipid in nanoparticles is approximately 1% by weight, approximately 2% by weight, approximately 3% by weight, approximately 4% by weight, approximately 5% by weight, approximately 10% by weight, approximately 15% by weight, approximately 20% by weight, approximately 25% by weight, or approximately 30% by weight.

In some embodiments, lipids are biocompatible oils. Suitable oils for use include plant oils and butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Oils may include one or more fatty acid groups or salts thereof. In some embodiments, a fatty acid group is digestible, long chain (e.g., C₈-C₅₀), substituted or unsubstituted hydrocarbons. In some embodiments, a fatty acid group is a C₁₀-C₂₀ fatty acid, C₁₅-C₂₀ fatty acid, or C₁₅-C₂₅ fatty acid or salt thereof. The fatty acid group can be unsaturated, monounsaturated, or polyunsaturated. In some embodiments, a double bond of an unsaturated fatty acid group is in the cis conformation. In some embodiments, a double bond of an unsaturated fatty acid is in the trans conformation.

In some embodiments, a fatty acid group is one or more of butyric, caproic, caprylic, capric, lauric, myristic, palmitic, stearic, arachidic, behenic, or lignoceric acid. In some embodiments, a fatty acid group is one or more of palmitoleic, oleic, vaccenic, linoleic, alpha-linolenic, gamma-linoleic, arachidonic, gadoleic, arachidonic, eicosapentaenoic, docosahexaenoic, or erucic acid. In some embodiments, the oil is a liquid triglyceride.

In some embodiments, a lipid is a steroid (e.g., cholesterol, bile acid), vitamin (e g vitamin E), phospholipid (e.g. phosphatidyl choline), sphingolipid (e.g. ceramides), or lipoprotein (e.g. apolipoprotein). In some embodiments, a lipid is a lipid-like material (also called lipidoid). See Akinc, et al., Nat Biotechnol., 2008: 26 (5): 561-9; Love, et al., Proc Natl Acad Sci USA. 2010: 107 (5): 1864-9; and Whitehead, et al., Nat. Commun., 2014:5:4277.

In certain embodiments, the lipid is phosphatidylcholine, lipid A, cholesterol, dolichol, sphingosine, sphingomyelin, ceramide, glycosylceramide, cerebroside, sulfatide, phytosphingosine, phosphatidyl-ethanolamine, phosphatidylglycerol, phosphatidylinositol, phosphatidylserine, cardiolipin, phosphatidic acid, and/or lyso-phophatides.

In some embodiments, nanoparticle-stabilized liposomes are used to deliver the disclosed compositions. By allowing small charged nanoparticles (1 nm-30 nm) to adsorb on liposome surface, liposome-nanoparticle complexes have not only the merits of bare liposomes, but also tunable membrane rigidity and controllable liposome stability. When small charged nanoparticles approach the surface of liposomes carrying either opposite charge or no net charge, electrostatic or charge-dipole interaction between nanoparticles and membrane attracts the nanoparticles to stay on the membrane surface, being partially wrapped by lipid membrane. This induces local membrane bending and globule surface tension of liposomes, both of which enable tuning of membrane rigidity. Adsorbed nanoparticles form a charged shell which protects liposomes against fusion, thereby enhancing liposome stability. In certain embodiments, small nanoparticles are mixed with liposomes under gentle vortex, and the nanoparticles stick to liposome surface spontaneously.

Lipid-Polymer Delivery Vehicles: In some embodiments, nanoparticles include one or more polymers associated covalently, or non-covalently with one or more lipids, preferably phospholipids. In some embodiments, a polymeric matrix can be surrounded by a lipid coating layer (e.g., liposome, lipid monolayer, micelle, etc.). The lipid monolayer shell can include an amphiphilic compound. In another embodiment, the amphiphilic compound is lecithin. The lipid monolayer can be stabilized.

Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and β-acyl-y-alkyl phospholipids. In a particular embodiment, an amphiphilic component that can be used to form an amphiphilic layer is lecithin, and, in particular, phosphatidylcholine. Lecithin is an amphiphilic lipid and, as such, forms a phospholipid bilayer having the hydrophilic (polar) heads facing their surroundings, which are oftentimes aqueous, and the hydrophobic tails facing each other. Lecithin has an advantage of being a natural lipid that is available from, e.g., soybean, and already has FDA approval for use in other delivery devices.

Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine, incorporated at a ratio of between 0.01-60 (weight lipid/w polymer) or between 0.1-30 (weight lipid/w polymer). Synthetic phospholipids with asymmetric acyl chains (e.g., with one acyl chain of 6 carbons and another acyl chain of 12 carbons) may also be used.

By covering the polymeric nanoparticles with a thin film of small molecule amphiphilic compounds, the nanoparticles have merits of both polymer- and lipid-based nanoparticles, while excluding some of their limitations. The amphiphilic compounds form a tightly assembled monolayer around the polymeric core. This monolayer effectively prevents the carried agents from freely diffusing out of the nanoparticle, thereby enhancing the encapsulation yield and slowing drug release. Moreover, the amphiphilic monolayer also reduces water penetration rate into the nanoparticle, which slows the hydrolysis rate of the biodegradable polymers, thereby increasing particle stability and lifetime.

In some embodiments, the nanoparticle include a polymeric matrix, wherein the polymeric matrix includes a lipid-terminated polymer such as polyalkylene glycol and/or a polyester. In some embodiments, the nanoparticle includes an amphiphilic lipid-terminated polymer, where a cationic and/or an aniotic lipid is conjugated to a hydrophobic polymer. In one embodiment, the polymeric matrix includes lipid-terminated PEG. In some embodiments, the polymeric matrix includes lipid-terminated copolymer. In another embodiment, the polymeric matrix includes lipid-terminated PEG and PLGA. In one embodiment, the lipid is 1,2 distearoyl-sn-glycero-3-phosphoethanolamine (DSPE), and salts thereof. In a preferred embodiment, the polymeric matrix includes DSPE-terminated PEG. The lipid-terminated PEG can then, for example, be mixed with PLGA to form a nanoparticle.

In some embodiments, long-circulating, optionally cell-penetrating, and stimuli-responsive nanoparticles for effective in vivo delivery of therapeutic, prophylactic and/or diagnostic agents are used. In certain embodiment, the NPs are made of an amphiphilic polymer, most preferably a PEGylated polymer, which shows a response to a stimulus such as pH, temperature, or light, such as an ultra pH-responsive characteristic with a pKa close to the endosomal pH (6.0-6.5) (Wang Y et al., Nat Mater, 13, 204-212 (2014)). The polymer may include a targeting or cell penetrating or adhesion molecule such as peptide iRGD.

### Kits

The compositions described herein can be packaged in suitable containers labeled, for example, for use detecting off-target genome editing in vitro or in vivo. The kit can contain a gene editing agent, e.g. Clustered Regularly Interspaced Short Palindromic Repeat (CRISPR)-associated endonuclease, a guide RNA (gRNA), an inhibitor of DNA repair, a delivery vehicle and combinations thereof.

Accordingly, packaged products (e.g., sterile containers containing one or more of the compositions described herein and packaged for storage, shipment, or sale at concentrated or ready-to-use concentrations) and kits, including at least one composition of the invention, and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers pharmaceutical carriers or other control reagents.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

### EXAMPLES

### Example 1: Improving the Sensitivity of In Vivo CRISPR Off-Target Detection

Discovery of off-target CRISPR-Cas genome editing in patient-derived cells and animal models is crucial for therapeutic applications, but exhibits low sensitivity due to reliance on detection of transient repair factor MRE11 binding events at Cas9-targeted sites. It is demonstrated herein, how inhibition of DNA-dependent protein kinase catalytic subunit (DNA-PKcs) accumulates MRE11 at Cas9-targeted sites, increasing the sensitivity of off-target detection up to over 5-fold in cell lines, induced pluripotent stem cells, and mice. Termed herein, DISCOVER-Seq+, was demonstrated to be the most sensitive method to-date for discovery of off-target genome editing in vivo.

### Methods

### Cell Culture

HEK293T cells (ATCC® CRL-3216™) and K562 cells (ATCC® CCL-243™) were cultured at 37° C. under 5% CO₂ in Dulbecco's Modified Eagle's Medium (DMEM, Corning) supplemented with 10% FBS (Clontech), 100 units/mL penicillin, and 100 μg/mL streptomycin (DMEM complete). Cells were tested every month for mycoplasma.

A human induced pluripotent stem cell (hiPSC), WTC11 cell line (Kreitzer et al., 2013), was used for all iPS cell experiments in this study. The guidelines of Johns Hopkins Medical Institute were followed for the use of this hiPSC line. Briefly, frozen WTC11 cells were first thawed in 37° C. water bath and washed in Essential 8 Medium (E8; Thermo Fisher Scientific, #A1517001) by centrifugation. After resuspension, WTC cells were plated onto a 6 cm cell culture dish pre-coated with human embryonic cell (hES cell)-qualified matrigel (1:100 dilution, Corning #354277). Plate coating should be performed for at least 2 h. Subsequently, 10 μM ROCK inhibitor (Y-27632: STEMCELL, #72308) was supplemented into the E8 medium to promote cell growth and survival. For subculture, WTC11 cells were dissociated from the plate using accutase (Sigma, #A6964) and passaged every 2 days. WTC11 cells were maintained in an incubator at 37° C. with 5% CO₂.

### Mouse Husbandry

All mouse studies were carried out in accordance with guidelines and approval of the Johns Hopkins University Animal Care and Use Committee (Protocol #MO20M274). Male C57BL/6J mice (The Jackson Laboratory, ME, USA) were housed in a facility with 12-hour light/12-hour dark cycle at 22±1° C. and 40±10% humidity. Teklad global 18% protein rodent diet and tap water were provided ad libitum.

### Immunofluorescence and Imaging by STED Microscopy

U2OS cells stably expressing Cas9-EGFP cells were seeded onto 35-mm, glass bottom dishes and transfected with mtgRNAs for 12-24 hrs. Cleavage was activated by UV light for 1 minute. To fix cells, 4% of pre-warmed paraformaldehyde in 1×PBS was used for 10 min. After rinsing 3 times with 1×PBS, cell membrane permeabilization was done with Triton-X used for 10 min. Then, cells 2% w/v BSA in 1×PBS was used for blocking for 1 hr and at room temperature. The primary antibodies, mouse anti-BRCA1 (D-9 sc-6954 Santa Cruz) and anti-53bpl (Abcam, ab172580), were diluted (1:500) in 1×PBS and directly added into the imaging dish. After 1 hr incubation, the primary antibody was removed, and the sample was washed with 1×PBS three times. The samples were then incubated for 30 minutes with the secondary antibodies Alexa-594 and Atto-647N diluted (1:1000) in 1×PBS. Finally, the sample was rinsed three times and mounted with Prolong Diamond mounting media (Thermo Fisher Scientific) overnight.

All STED images were obtained using a home-built two-color STED microscope (Han and Ha, 2015: Ma and Ha, 2019). In short, a femtosecond laser beam with a repetition rate of 80 MHz from a Ti: Sapphire laser head (Mai Tai HP, Spectra-Physics) is split into two parts: one part produces an excitation beam coupled into a photonic crystal fiber (Newport) for wide-spectrum light generation. The beam is further filtered by a frequency-tunable acoustic optical filter (AA Opto-Electronic) for multi-color excitation. The other part of the laser pulse is temporally stretched to ˜300 ps (with two 15-cm-long glass rods and a 100-m long polarization-maintaining single-mode fiber, OZ optics), collimated and expanded, and wave-front modulated with a vortex phase plate (VPP-1, RPC photonics). This modulation produces a hollow STED spot generation to de-excite the fluorophores at the periphery of the excitation focus, thus improving the lateral resolution. The STED beam is set at 765 nm with a power of 120 mW at the back focal plane of the objective (NA=1.4 HCX PL APO 100×, Leica), and the excitation wavelengths are set as 594 nm and 650 nm for imaging Alexa-594 and Atto-647N labeled targets, respectively. Two avalanche photodiodes detect the fluorescent photons (SPCM-AQR-14-FC, Perkin Elmer). The images are obtained by scanning a piezo-controlled stage (Max311D, Thorlabs) controlled with the Imspector data acquisition program.

Electroporation of Cas9 RNP into Cell Lines and iPSCs

crRNA and tracrRNA sequences are in Table 1 below. 2 μL of 100 μM crRNA was mixed with 2 μL of 100 μM tracrRNA (Integrated DNA Technologies) and heated to 95° C. for 5 min in a thermocycler, then allowed to cool on benchtop for 5 min. To form the RNP complex, 3 μL of 10 μg/μL (˜66 μM) of purified Cas9 was mixed with the annealed 4 μL 50 μM cr: tracrRNA, then 8 μL of dialysis buffer (20 mM HEPES pH 7.5, and 500 mM KCl, 20% glycerol) was mixed in for a total of 15 μL. This solution as incubated for 20 min at room temperature to allow for RNP formation.

TABLE 1

| Name | Sequence (5′ to 3′ ) |
|---|---|
| tracrRNA | AGCAUAGCAAGUUAAAAUAAGGCUAGUCCG |
| (SEQ ID NO: 1) | UUAUCAACUUGAAAAAGUGGCACCGAGUCG |
| | GUGCUUU |
| IDT_FANCFsite2 | GCUGCAGAAGGGAUUCCAUGGUUUUAGAGC |
| (SEQ ID NO: 2) | UAUGCU |
| IDT_HEKsite4 | GGCACUGCGGCUGGAGGUGGGUUUUAGAGC |
| (SEQ ID NO: 3) | UAUGCU |
| IDT_VEGFAsite2 | GACCCCCUCCACCCCGCCUCGUUUUAGAGC |
| (SEQ ID NO: 4) | UAUGCU |
| IDT_VEGFAsite3 | GGUGAGUGAGUGUGUGCGUGGUUUUAGAGC |
| (SEQ ID NO: 5) | UAUGCU |

HEK293T cells were maintained to a confluency of ˜90% prior to electroporation. 12 million cells were trypsinized with 5 min incubation in the incubator, then 1:1 of DMEM complete was added to inactivate trypsin. This mixture was centrifuged (3 min, 200×g), supernatant removed, followed by resuspension of the cell pellet in 1 mL PBS, centrifugation (3 min, 200×g), and finally complete removal of supernatant. 90 μL of nucleofection solution (16.2 μL of Supplement solution mixed with 73.8 μL of SF solution from SF Cell Line 4D-NUCLEOFECTOR™ X Kit L) (Lonza) was mixed thoroughly with the cell pellet. The 15 μL RNP solution was mixed in along with 2 μL of Cas9 Electroporation Enhancer (Integrated DNA Technologies). The entirety of the final solution (approximately 125 μL) was transferred to one well of a provided cuvette rated for 100 μL. Electroporation was then performed according to the manufacturer's instructions on the 4D-NUCLEOFECTOR™ Core Unit (Lonza) using code CA-189. Some white residue may appear in the cell mixture after electroporation, but that is completely normal. A total of 400 μL of DMEM complete was used to completely transfer the cells out of the cuvette, before plating to culture wells pre-coated with 1:100 collagen. A minimum of 4 million cells are used for each ChIP. For time-resolved experiments, this means one electroporation equates to 3 samples.

For WTC-11 iPSCs, cells were dissociated from the plate using accutase (Sigma, #A6964). Electroporation was performed using the Lonza P3 Primary Cell 4D-NUCLEOFECTOR™ X Kit L using code CA-137, on 10 million cells, and using 65 μL of the P3 solution mixture with EP enhancer per electroporation cuvette (compared to 90 μL of comparable SF solution mixture for HEK293T cells). After electroporation, cells were resuspended in E8 medium supplemented with 10 μM ROCK inhibitor (Y-27632; STEMCELL, #72308), and plated onto a 10 cm cell culture dish pre-coated with human embryonic cell (hES cell)-qualified matrigel (1:100 dilution, Corning #354277) for at least 2 hours.

To expose cells to DNA repair inhibitors, they were added to the culture media at a final concentration of 1 μM KU-60648 (1:2500 of 2.5 mM KU-60648), 20 UM Nu7026 (1:500 of 10 mM Nu7026), 10 μM Ku-55933 (1:10000 of 100 mM KU-55933), 1 μM Scr7 (1:10000 of 10 mM Scr7), or 10 μM Olaparib (1:1000 of 10 mM Olaparib). All stock solutions of drug were diluted in DMSO.

Adenovirus and DNA-PKcs Inhibitor Delivery into Mice

For in vivo gene delivery, 8-10 week old mice were anesthetized with 2.5% isofluorane/oxygen mixture. Mice received a single retro-orbital injection of 1×10⁹ infectious adenoviral particles (Ad-Cas9-U6-mPCSK9-sgRNA) in 100 μL sterile saline. Immediately following, mice received intraperitoneal delivery of KU-60648 dosed at 25 mg/kg (or vehicle only) in 100 μL of citrate buffer, or 100 μL of citrate buffer vehicle. Mice received a dose of KU-60648 or vehicle every 12 hrs via intraperitoneal injection.

Extraction of Mouse Liver into Cell Suspension

At the experimental endpoint of 12 h, mice were anesthetized with isofluorane and euthanized via cervical dislocation. Liver tissue was harvested, washed 3× in 2 mL PBS with 1× protease inhibitor (Halt™ Protease Inhibitor Cocktail, Thermo), then disrupted the tissue in 1 mL PBS with 1× protease inhibitor using a loose-fitting Dounce homogenizer. For MRE11 ChIP-seq, homogenized tissue was placed on ice and used immediately.

### DISCOVER-Seq+/DISCOVER-Seq/MRE11 ChIP-Seq

The protocol was adapted from previous literature (Wienert et al., 2019) and describes the reagents for one MRE11 ChIP-seq experiment.

For adherent cells, approximately 10 million cells were gently rinsed with room temperature PBS, washed off the plate using 10 mL DMEM with assistance from pipette squirts and cell scraper, then transferred to a 15 mL Falcon tube. For suspension cells, approximately 10 million cells were transferred to a 15 mL Falcon tube, spun down 200×g for 1 min, decanted, then resuspended with 10 mL DMEM. 721 μL of 16% formaldehyde(methanol-free) was added and the tube was mixed by inversion in room temperature—7 min for WTC-11 iPSCs, 12 min for HEK293T cells, or 15 min for K562 cells. For mouse liver, 300 μL of Dounce homogenized mouse liver was diluted into 10 mL PBS. 721 μL of 16% formaldehyde(methanol-free) was added and mixed by inversion in room temperature for 10 min.

Afterwards, 750 μL of 2 M glycine was added to quench the formaldehyde. Cells were spun down with 1,200×g at 4° C. for 3 min, then washed with ice-cold PBS twice, spinning down with the same centrifugation conditions. Pellet can be decanted, flash-frozen, then stored in −80° C. for later use. Cells were then resuspended in 4 mL lysis buffer LB1 (50 mM HEPES, 140 mM NaCl, 1 mM EDTA, 10% glycerol, 0.5% Igepal CA-630, 0.25% Triton X-100, pH to 7.5 using KOH, add 1× protease inhibitor right before use) for 10 min at 4° C. then spun down 2,000×g at 4° C. for 3 min. The supernatant was decanted. Cells were then resuspended in 4 mL LB2 (10 mM Tris-HCl pH 8, 200 mM NaCl, 1 mM EDTA, 0.5 mM EGTA, pH to 8.0 using HCl, add 1× protease inhibitor right before use) for 5 min at 4° C., spun down with the same protocol, and the supernatant decanted. Cells were then resuspended in 1.5 mL LB3 (10 mM Tris-HCl pH 8, 100 mM NaCl, 1 mM EDTA, 0.5 mM EGTA, 0.1% Na-Deoxycholate, 0.5% N-lauroylsarcosine, pH to 8.0 using HCl, add 1× protease inhibitor right before use) and transferred to 2 mL Eppendorf tubes for sonication with 50% amplitude, 30 s ON, 30 s OFF for 12 min total time (Fisher 150E Sonic Dismembrator). Sample was spun down with 20,000×g at 4° C. for 10 min, and supernatant was transferred to 1.5 mL LB3 in a 15 mL falcon tube. 300 μL of 10% Triton X-100 was added, and the entire solution was well mixed by gentle inversion.

Beads pre-loaded with antibodies were prepared before cell harvesting. 50 μL Protein A beads (Thermo Fisher) were used per IP and transferred to a 2 mL Eppendorf tube on a magnetic stand. Beads were washed twice with blocking buffer BB (0.5% BSA in PBS), then resuspended in 100 μL BB per IP. 4 μL of MRE11 antibody (Novus NB100-142) per IP was added and placed on rotator for 1-2 hours. Right before IP, the 2 mL tube was placed on a magnetic rack and washed 3× with BB, before resuspending in 50 μL BB per EP. 50 μL of beads in BB were transferred to each IP and placed in 4° C. rotator for 6+hours.

Samples were transferred to 2 mL Eppendorf tubes on a magnetic stand, washed 6× with 1 mL RIPA buffer (50 mM HEPES, 500 mM LiCl, 1 mM EDTA, 1% Igepal CA-630, 0.7% Na-Deoxycholate, pH to 7.5 using KOH), then washed 1× with 1 mL TBE buffer (20 mM Tris-HCl pH 7.5, 150 mM NaCl), before decanting. Beads containing ChIP-ed DNA were mixed with 70 μL elution buffer EB (50 mM Tris-HCl pH 8.0, 10 mM EDTA, 1% SDS) and incubated 65° C. for 6+ hours. 40 μL of TE buffer was mixed to dilute the SDS, followed by 2 μL of 20 mg/mL RNaseA (New England BioLabs) for 30 min at 37° C. 4 μL of 20 mg/mL Proteinase K (New England BioLabs) was added and incubated for 1 hours at 55° C. The genomic DNA was column purified (Qiagen) and eluted in 35 μL nuclease free water.

Oligo sequences for library preparation are in Table 3. End-repair/A-tailing was performed on 17 μL of ChIPed DNA using NEBNextR Ultra™ II End Repair/dA-Tailing Module (New England BioLabs), followed by ligation (MNase_F/MNase_R) with T4 DNA Ligase (New England BioLabs). 13 cycles of PCR using PE_i5 and PE_i7XX primer pairs were performed for MRE11 ChIP samples to amplify sequencing libraries. Samples were pooled, quantified with QuBit (Thermo), Bioanalyzer (Agilent) and qPCR (BioRad).

TABLE 3

| Name | Sequence (5′ to 3′ ) |
|---|---|
| MNase_F | /5 Phos/GATCGGAAGAGCACACGTCT |
| (SEQ ID | |
| NO: 24) | |
| MNase_R | ACACTCTTTCCCTACACGACGCTCTTCCGATC*T |
| (SEQ ID | |
| NO: 25) | |
| PE_i5 | AATGATACGGCGACCACCGAGATCTACACTCTTTCCCTACACGACGCTCTTCCGATC*T |
| (SEQ ID | |
| NO: 26) | |
| PE_i701 | CAAGCAGAAGACGGCATACGAGATCGTGATGTGACTGGAGTTCAGACGTGTGCTCTTCCGATC*T |
| (SEQ ID | |
| NO: 27) | |
| PE_i702 | CAAGCAGAAGACGGCATACGAGATACATCGGTGACTGGAGTTCAGACGTGTGCTCTTCCGATC*T |
| (SEQ ID | |
| NO: 28) | |
| PE_i703 | CAAGCAGAAGACGGCATACGAGATGCCTAAGTGACTGGAGTTCAGACGTGTGCTCTTCCGATC*T |
| (SEQ ID | |
| NO: 29) | |
| PE_i704 | CAAGCAGAAGACGGCATACGAGATTGGTCAGTGACTGGAGTTCAGACGTGTGCTCTTCCGATC*T |
| (SEQ ID | |
| NO: 30) | |
| PE_i705 | CAAGCAGAAGACGGCATACGAGATCACTGTGTGACTGGAGTTCAGACGTGTGCTCTTCCGATC*T |
| (SEQ ID | |
| NO: 31) | |
| PE_i706 | CAAGCAGAAGACGGCATACGAGATATTGGCGTGACTGGAGTTCAGACGTGTGCTCTTCCGATC*T |
| (SEQ ID | |
| NO: 32) | |
| PE_i707 | CAAGCAGAAGACGGCATACGAGATGATCTGGTGACTGGAGTTCAGACGTGTGCTCTTCCGATC*T |
| (SEQ ID | |
| NO: 33) | |
| PE_i708 | CAAGCAGAAGACGGCATACGAGATTCAAGTGTGACTGGAGTTCAGACGTGTGCTCTTCCGATC*T |
| (SEQ ID | |
| NO: 34) | |
| PE_i709 | CAAGCAGAAGACGGCATACGAGATCTGATCGTGACTGGAGTTCAGACGTGTGCTCTTCCGATC*T |
| (SEQ ID | |
| NO: 35) | |
| PE_i710 | CAAGCAGAAGACGGCATACGAGATAAGCTAGTGACTGGAGTTCAGACGTGTGCTCTTCCGATC*T |
| (SEQ ID | |
| NO: 36) | |
| PE_i711 | CAAGCAGAAGACGGCATACGAGATGTAGCCGTGACTGGAGTTCAGACGTGTGCTCTTCCGATC*T |
| (SEQ ID | |
| NO: 37) | |
| PE_i712 | CAAGCAGAAGACGGCATACGAGATTACAAGGTGACTGGAGTTCAGACGTGTGCTCTTCCGATC*T |
| (SEQ ID | |
| NO: 38) | |
| PE_i713 | CAAGCAGAAGACGGCATACGAGATATCAGTGTGACTGGAGTTCAGACGTGTGCTCTTCCGATC*T |
| (SEQ ID | |
| NO: 39) | |
| PE_i714 | CAAGCAGAAGACGGCATACGAGATAGGAATGTGACTGGAGTTCAGACGTGTGCTCTTCCGATC*T |
| (SEQ ID | |
| NO: 40) | |
| PE_i715 | CAAGCAGAAGACGGCATACGAGATATTCCGGTGACTGGAGTTCAGACGTGTGCTCTTCCGATC*T |
| (SEQ ID | |
| NO: 41) | |
| PE_i716 | CAAGCAGAAGACGGCATACGAGATCCACTCGTGACTGGAGTTCAGACGTGTGCTCTTCCGATC*T |
| (SEQ ID | |
| NO: 42) | |
| PE_i717 | CAAGCAGAAGACGGCATACGAGATCGATTAGTGACTGGAGTTCAGACGTGTGCTCTTCCGATC*T |
| (SEQ ID | |
| NO: 43) | |
| PE_i718 | CAAGCAGAAGACGGCATACGAGATCTTCGAGTGACTGGAGTTCAGACGTGTGCTCTTCCGATC*T |
| (SEQ ID | |
| NO: 44) | |
| PE_i719 | CAAGCAGAAGACGGCATACGAGATGAATGAGTGACTGGAGTTCAGACGTGTGCTCTTCCGATC*T |
| (SEQ ID | |
| NO: 45) | |
| PE_i720 | CAAGCAGAAGACGGCATACGAGATGCGGACGTGACTGGAGTTCAGACGTGTGCTCTTCCGATC*T |
| (SEQ ID | |
| NO: 46) | |
| PE_i721 | CAAGCAGAAGACGGCATACGAGATGGAACTGTGACTGGAGTTCAGACGTGTGCTCTTCCGATC*T |
| (SEQ ID | |
| NO: 47) | |
| PE_i722 | CAAGCAGAAGACGGCATACGAGATTAGTTGGTGACTGGAGTTCAGACGTGTGCTCTTCCGATC*T |
| (SEQ ID | |
| NO: 48) | |
| PE_i723 | CAAGCAGAAGACGGCATACGAGATTCGGGAGTGACTGGAGTTCAGACGTGTGCTCTTCCGATC*T |
| (SEQ ID | |
| NO: 49) | |
| PE_i724 | CAAGCAGAAGACGGCATACGAGATTCTGAGGTGACTGGAGTTCAGACGTGTGCTCTTCCGATC*T |
| (SEQ ID | |
| NO: 24) | |

Cell line samples were sequenced on a NextSeq 500 (Illumina) using paired 2×36 bp reads. Mouse liver samples were sequenced on a DNBSEQ PE100 (BGI) using paired 2×50 bp reads. All ChIP-seq raw reads in FASTQ format and processed alignments in BAM format are uploaded to Sequence Read Archive (SRA) under BioProject accession PRJNA801688.

Reads were demultiplexed after sequencing using bcl2fastq. Paired-end reads were aligned to hg38, hg19, or mm10 using bowtie2. To ensure fair comparison between DISCOVER-Seq+ (with DNA-PKcs inhibitor) and DISCOVER-Seq (without inhibitor), equal numbers of sequencing reads were obtained by subsetting for each set of samples. Samtools was used to filtered for mapping quality>=25, remove singleton reads, convert to BAM format, remove potential PCR duplicates, and index BAM-formatted output files. The software that coordinates these steps as well as performs subsequent analyses are open source (https://github.com/rogerzou/D) SeqPlus).

BLENDER (Wienert et al., 2019) (https://github.com/staciawyman/blender) was used to determine Cas9 off-target sites, outputting a curated list of all off-target sites with corresponding visualization. A more sensitive cutoff threshold of 2 (−c 2) was used for all samples except a threshold of 3 (−c 3) for the merged PCSK9 samples.

### CRISPR-Cas9 or Cas1 2a Editing of Primary Human T-Cells

Engineered T cells expressing a TP53 R175H: HLA-A*02:01-specific TCR under control of a EF1-alpha promoter were generated via CRISPR-Cas-mediated homology directed repair (HDR) electroporation as follows. Nucleotide sequences of the TCR of interest, promoter, and homology arms for the TRAC gene locus were generated by de-novo gene synthesis (GeneArt). HDR template DNA (HDRT) was generated by amplification from a plasmid template using the Q5 High-Fidelity 2X Master Mix (New England BioLabs) with primers containing truncated Cas9 target sequences (IDT). Amplicon DNA was purified with 1×AMPure beads (Beckman Coulter), eluted in water, and quantified. Purified PCR products were analyzed by agarose gel electrophoresis to assess correct amplicon size and purity. T cells were isolated by negative selection using immunomagnetic cell separation (EasySep Human T Cell Isolation Kit) from cryopreserved healthy donor peripheral blood mononuclear cells collected via leukapheresis. Purified CD3+ T cells were activated with Dynabeads™ Human T-Activator CD3/CD28 (ThermoFisher) at a 1:2 bead-to-cell ratio in RPMI-1640 (ATCC) supplemented with 10% fetal bovine serum (HyClone Defined), 100 units/mL Penicillin (Gibco), 100 μg/mL Streptomycin (Gibco), 100 IU/mL recombinant human IL-2 (Proleukin, Prometheus Laboratories) and 5 ng/mL recombinant human IL-7 (BioLegend) at 37 C, 5% CO₂. After 48 hours and prior to electroporation, CD3/CD28 beads were removed with a magnet. Cas9 ribonucleoprotein (RNP) targeting TRAC (AGAGTCTCTCAGCTGGTACA) or Cpf1 (Cas12a) RNP targeting a juxtaposed nucleotide sequence in TRAC (GAGTCTCTCAGCTGGTACAC) were assembled by mixing the appropriate sgRNA (IDT) with either Alt-R S.p. Cas9 nuclease V3 (IDT) or Alt-R A.s. Cas12a (Cpf1) Ultra nuclease (IDT) and matching ssDNA Electroporation Enhancer (IDT) and incubating the mixture at room temperature for 15 minutes. RNPs were mixed with 0.5 μg of the same HDRT and incubated for 5 minutes at room temperature. To edit activated T cells, 20 μL of T cells were resuspended in P3 buffer at 5×10⁷ cells/mL (Lonza) and added to the electroporation mixture. Electroporation was performed with a 4D-Nucleofector X Unit (Lonza) in 16-well cuvettes using pulse code EH115. After electroporation, T cells were recovered by immediately adding 80 μL of warm, cytokine-free T cell media to the cuvettes and incubation at 37° C. for 15 minutes. Then, T cells were diluted in T cell growth media containing 100 IU/mL recombinant human IL-2 and 5 ng/ml recombinant human IL-7 in the presence of [inhibitor, concentration] or vehicle (DMSO) and incubated for 12 hours at 37 C, 5% CO₂. A fraction of electroporated T cells for each condition was maintained in T cell growth media with human IL-2 and IL-7 for X days prior to analysis for gene editing rates and surface expression of TP53 R175H: HLA-A*02:01-specific TCR by flow cytometry.

### High Throughput Sequencing of Genomic DNA Samples

Genomic DNA was extracted using the DNeasy Blood & Tissue Kit (Qiagen 69504) following manufacturer instructions. Approximately 1 million cells were used from cell lines and iPSCs. Approximately 10-20 μL of mouse liver cell suspension was used out of 1.5 mL total, and the genome extraction protocol included the Buffer ATL step since this sample is tissue.

Genomic DNA samples were amplified with PCR using Q5 Hot Start High-Fidelity 2X Master Mix (New England BioLabs M0494). Primer pairs for all sequences are listed in Table 2 below. For example, the primer set for amplifying around the FANCF site 2 on-target site is NGS_FANCFs2_ON_F and NGS_FANCFs2_ON_R. After amplicon PCR, cleanup was performed using 1.4×AMPure XP (Beckman Coulter A63881) following the manufacturer's instructions. Dual-indexing PCR was performed using KAPA HiFi HotStart ReadyMix (Roche 07958935001) and PCR cleanup was performed using 1×AMPure XP. Samples were quantified using QuBit (Thermo Fisher Scientific), pooled, diluted, and loaded onto a MiSeq (Illumina). Sequencing was performed with the following number of cycles “151|8|8|151” with the paired-end Nextera sequencing protocol.

TABLE 2

| Name | Sequence (5′ to 3′ ) |
|---|---|
| NGS_FANCFs2_ON-F | tcgtcggcagcgtcagatgtgtataagagacagAGGTGCTGACGTAGGTAGTG |
| (SEQ ID NO: 6) | |
| NGS_FANCFs2_ON-R | gtctcgtgggctcggagatgtgtataagagacagCGTATCATTTCGCGGATGTTC |
| (SEQ ID NO: 7) | |
| NGS_Index_F1 | AATGATACGGCGACCACCGAGATCTACACCTCTCTATTCGTCGGCAGCGTC |
| (SEQ ID NO: 8) | |
| NGS_Index_F2 | AATGATACGGCGACCACCGAGATCTACACTATCCTCTTCGTCGGCAGCGTC |
| (SEQ ID NO: 9) | |
| NGS_Index_F3 | AATGATACGGCGACCACCGAGATCTACACGTAAGGAGTCGTCGGCAGCGTC |
| (SEQ ID NO: 10) | |
| NGS_Index_F4 | AATGATACGGCGACCACCGAGATCTACACACTGCATATCGTCGGCAGCGTC |
| (SEQ ID NO: 11) | |
| NGS_Index_F5 | AATGATACGGCGACCACCGAGATCTACACAAGGAGTATCGTCGGCAGCGTC |
| (SEQ ID NO: 12) | |
| NGS_Index_F6 | AATGATACGGCGACCACCGAGATCTACACCTAAGCCTTCGTCGGCAGCGTC |
| (SEQ ID NO: 13) | |
| NGS_Index_F7 | AATGATACGGCGACCACCGAGATCTACACCGTCTAATTCGTCGGCAGCGTC |
| (SEQ ID NO: 14) | |
| NGS_Index_F8 | AATGATACGGCGACCACCGAGATCTACACTCTCTCCGTCGTCGGCAGCGTC |
| (SEQ ID NO: 15) | |
| NGS_Index_R1 | CAAGCAGAAGACGGCATACGAGATTCGCCTTAGTCTCGTGGGCTCGG |
| (SEQ ID NO: 16) | |
| NGS_Index_R2 | CAAGCAGAAGACGGCATACGAGATCTAGTACGGTCTCGTGGGCTCGG |
| (SEQ ID NO: 17) | |
| NGS_Index_R3 | CAAGCAGAAGACGGCATACGAGATTTCTGCCTGTCTCGTGGGCTCGG |
| (SEQ ID NO: 18) | |
| NGS_Index_R4 | CAAGCAGAAGACGGCATACGAGATGCTCAGGAGTCTCGTGGGCTCGG |
| (SEQ ID NO: 19) | |
| NGS_Index_R5 | CAAGCAGAAGACGGCATACGAGATAGGAGTCCGTCTCGTGGGCTCGG |
| (SEQ ID NO: 20) | |
| NGS_Index_R6 | CAAGCAGAAGACGGCATACGAGATCATGCCTAGTCTCGTGGGCTCGG |
| (SEQ ID NO: 21) | |
| NGS_Index_R7 | CAAGCAGAAGACGGCATACGAGATGTAGAGAGGTCTCGTGGGCTCGG |
| (SEQ ID NO: 22) | |
| NGS_Index_R8 | CAAGCAGAAGACGGCATACGAGATCAGCCTCGGTCTCGTGGGCTCGG |
| (SEQ ID NO: 23) | |

Sequencing reads were either demultiplexed automatically using MiSeq Reporter (Illumina) or with a custom Python script to individual FASTQ files. For indel calling, sequencing reads were scanned for exact matches to two 20-bp sequences that flank +/−20 bp from the ends of the target sequence. If no exact matches were found, the read was excluded from analysis. After additional filtering for an average quality score>20, an indel is defined as a sequence that differs in length from the reference length.

### Results

### Effects of DNA-PKcs Inhibition on the CRISPR-Mediated DNA Damage Response

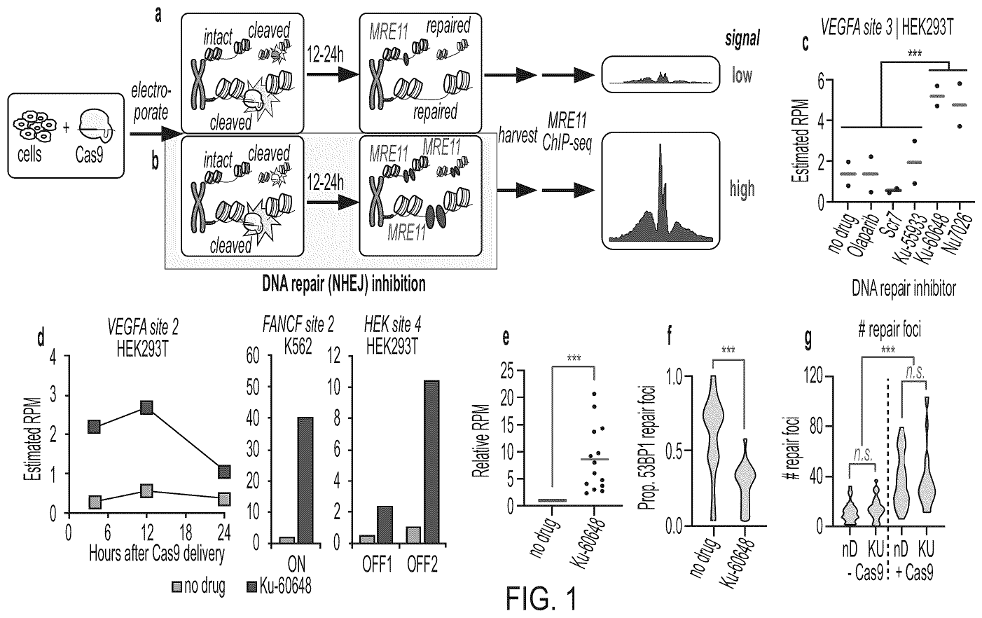

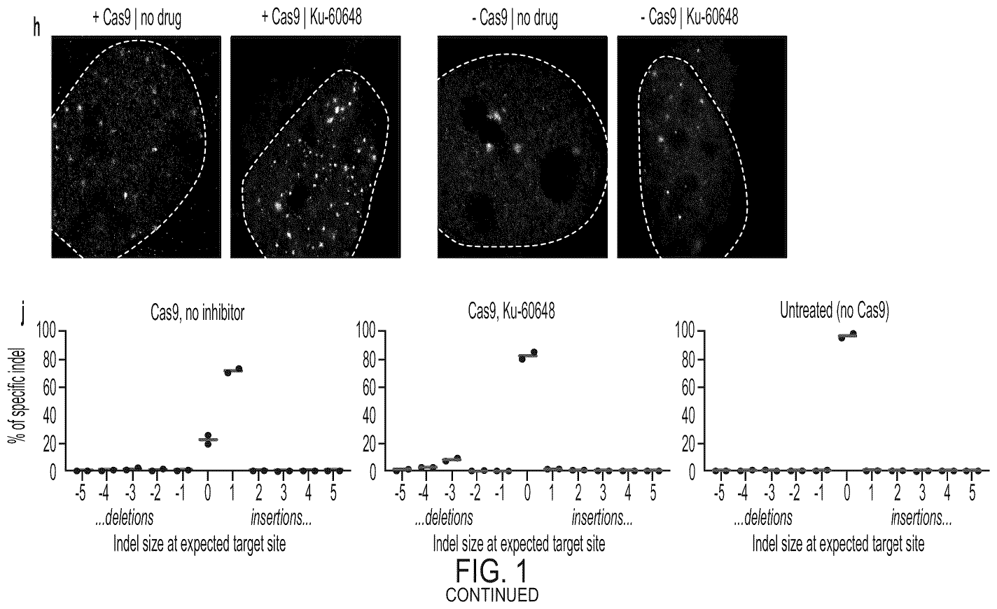

To identify inhibitors of DNA repair that can modulate MRE11 residence, we delivered Cas9 with guide RNA (gRNA) targeting VEGFA site 3 into HEK293T cells, exposed the cells to one of five DNA repair inhibitors, then measured MRE11 recruitment at the target site after 12 hours using ChIP with quantitative PCR (qPCR). Inhibition of Poly (ADP-ribose) polymerase (PARP) and ATM serine/threonine kinase (ATM) with Olaparib and Ku-55933, respectively, did not exhibit a clear effect, whereas DNA Ligase IV inhibition with Scr7 suppressed MRE11 recruitment (FIG. 1c) [20]. Notably, blocking non-homologous end-joining (NHEJ) by inhibiting DNA-PKcs using Ku-60648 [18,21] or Nu7026 significantly increased MRE11 recruitment at the target site (FIG. 1c). The effect of DNA-PKcs inhibition was consistent across multiple time points (4 hours, 12 hours, 24 hours), three other gRNAs (VEGFA site 2, HEK site 4, FANCF site 2), and/or another cell line (K562) (p<0.001) (FIG. 1d-e). These results suggest that blocking NHEJ with DNA-PKcs inhibition greatly boosts MRE11 residence at Cas9-targeted sites.

To better understand the effect of DNA-PKcs inhibition on repair of Cas9-mediated DNA damage, we used super-resolution stimulated emission depletion (STED) microscopy [23,24] to measure the localization of 53BP1 and BRCA1 foci after Cas9-induced DNA breaks. 53BP1 corresponds to activation of the NHEJ pathway, whereas BRCA1 is implicated in the MRE11-dependent homology directed repair (HDR) or microhomology-mediated end joining (MMEJ) [25,26]. Using a multi-target gRNA targeting over 100 locations [27], DNA-PKcs inhibition using Ku-60648 led to a reduction in 53BP1 foci relative to BRCA1, consistent with suppression of NHEJ in favor of HDR/MMEJ (FIG. 1f). Ku-60648 in the absence of Cas9 did not change the number of 53BP1 and BRCA1 foci detectable by SIM, suggesting that Ku-60648 alone does not induce DNA damage inside cells (FIG. 1g-i). Using a complementary assay, sequencing of insertion-deletion mutations (indels) after Cas9 targeting ACTB in the presence of Ku-60648 showed a reduction in editing, especially+1 insertions from NHEJ in favor of larger-3 deletions from MMEJ (FIG. 1j). Together, these results suggest that DNA-PKcs inhibition blocks the NHEJ repair pathway in favor of the MRE11-associated HDR and MMEJ pathways, thus boosting MRE11 residence.

MRE11 ChIP-Seq with DNA-PKcs Inhibition Increases Off-Target Detection Sensitivity

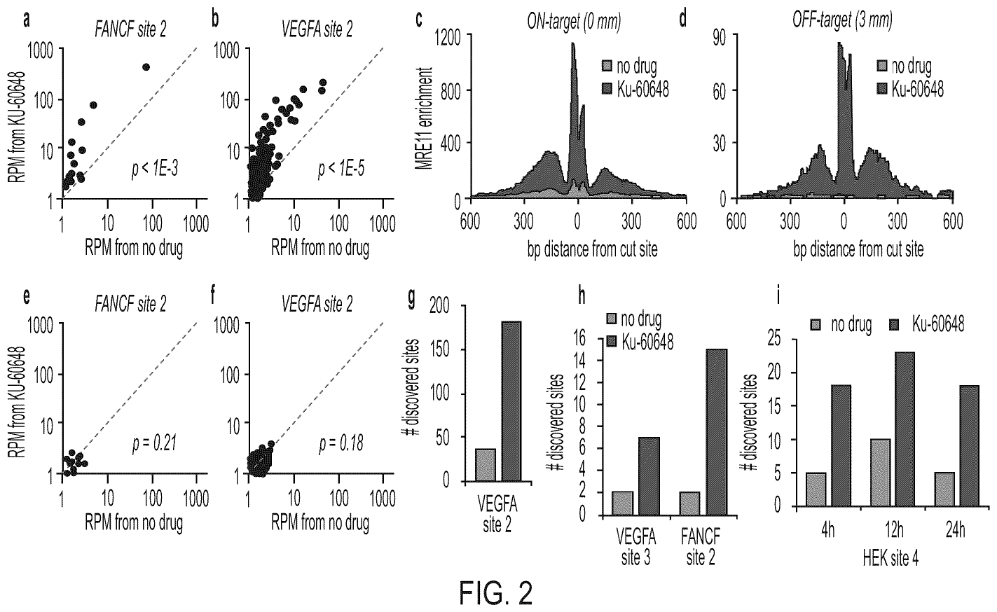

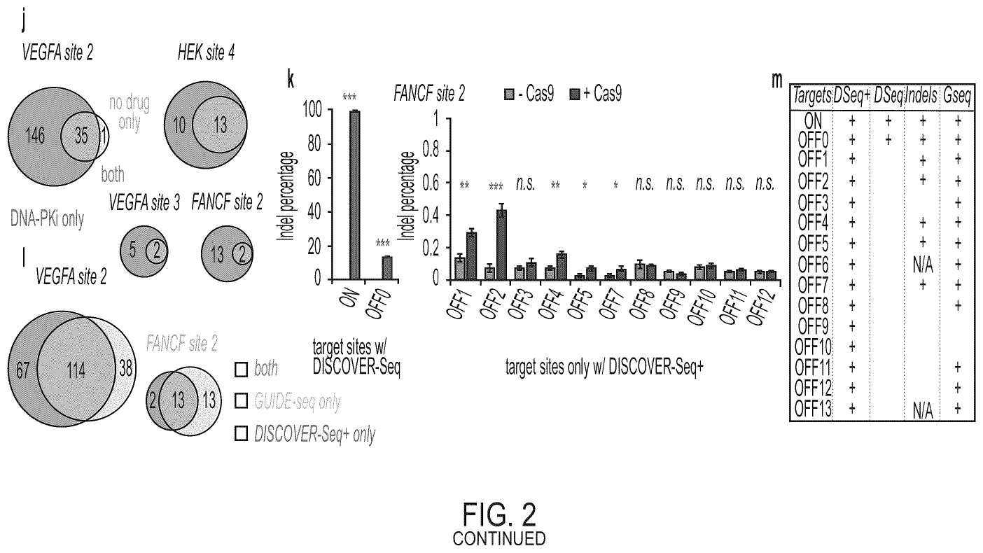

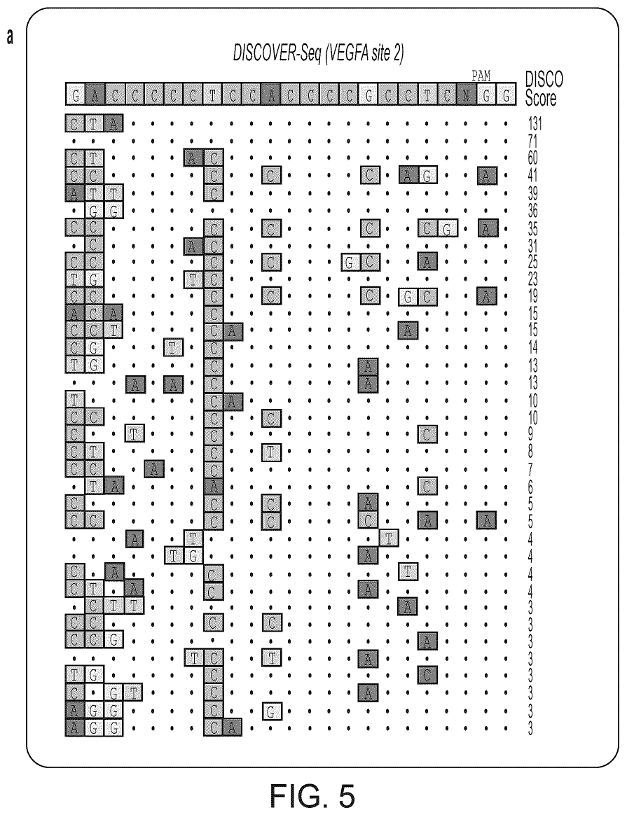

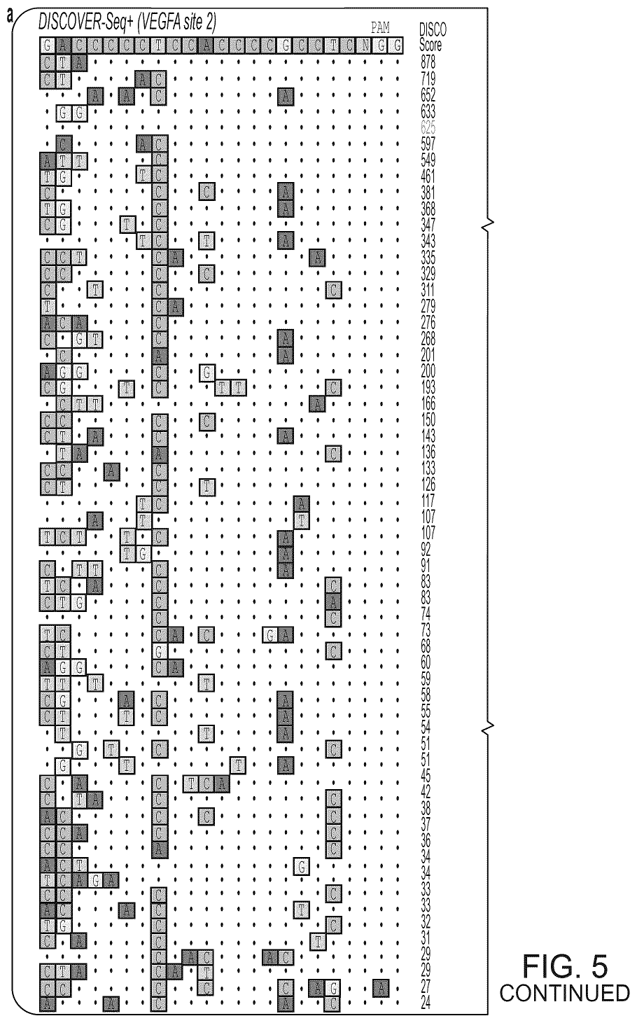

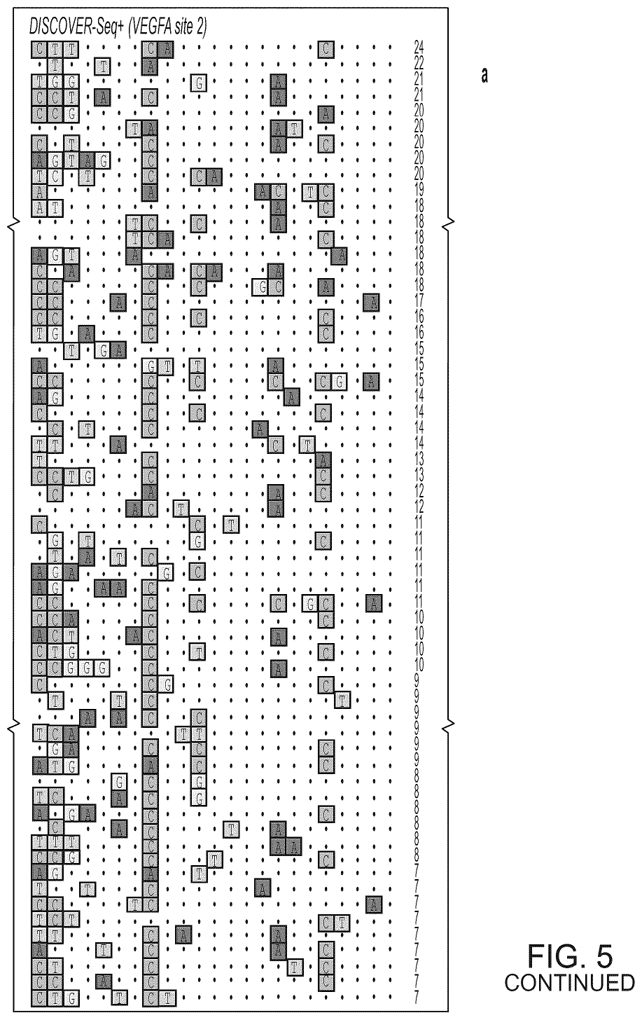

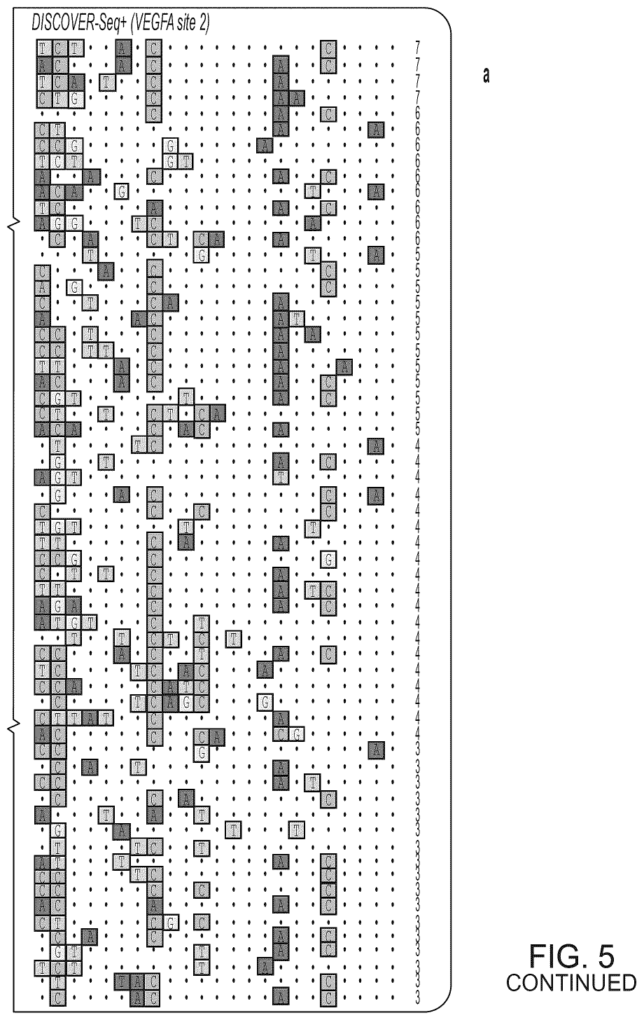

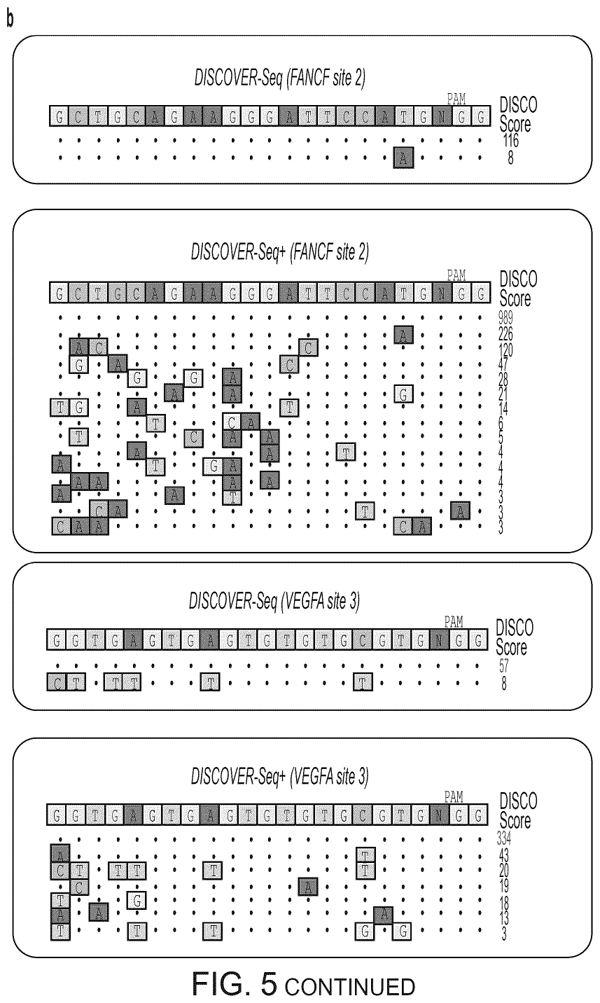

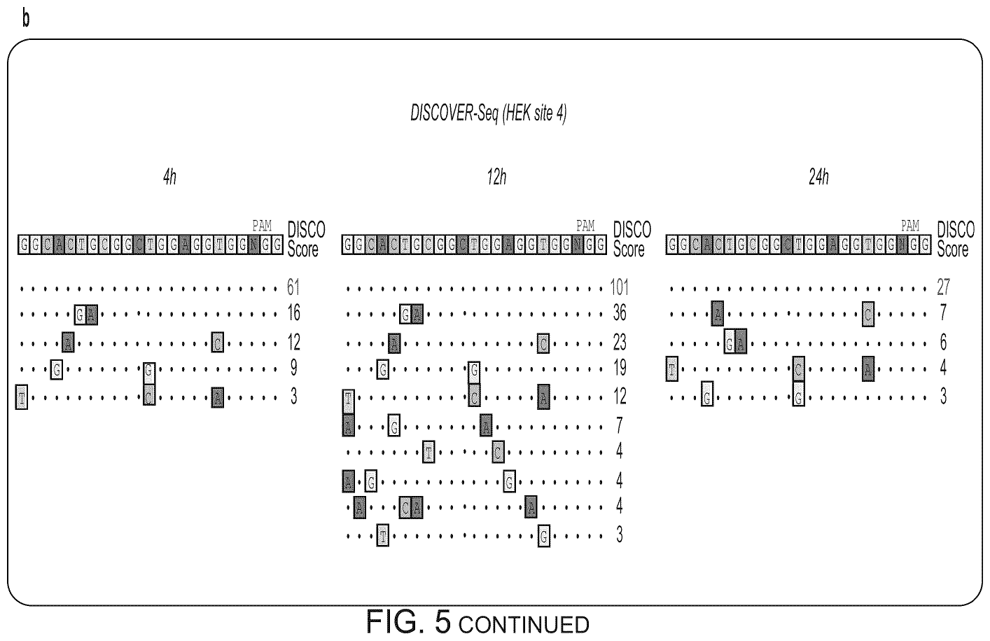

Next, we determined whether increased MRE11 residence with DNA-PKcs inhibition can improve the sensitivity of CRISPR off-target discovery. At 12 hours after Cas9 delivery into K562 cells, we performed ChIP-seq for MRE11, followed by the BLENDER bioinformatics pipeline to detect all Cas9 target sites genome-wide. Sequencing samples with or without DNA-PKcs inhibition were always normalized to the same number of reads for appropriate comparison. Treatment with Ku-60648 significantly increased MRE11 ChIP-seq enrichment at all discovered on- and off-target sites (p<1E-3) (FIG. 2a-d). MRE11 levels 10 kb away from the target sites did not significantly increase (p≥0.18), further supporting the lack of additional DNA damage caused by the inhibitor itself (FIG. 2e-f). 181 total Cas9 target sites were discovered for the VEGFA site 2 gRNA, which is an over 5-fold increase compared to the 36 sites discovered without DNA-PKcs inhibition (i.e., DISCOVER-Seq) (FIG. 2g; FIG. 5a). This final list of off-target sites was obtained after removing candidate off-target sites identified from control samples without Cas9, which likely correspond to false positives from the ChIP-seq process. Enhanced off-target discovery with DNA-PKcs inhibition was consistent across different gRNAs and multiple time points (FIG. 2h-i; FIG. 5b), and included the off-target sites identified using DISCOVER-Seq alone (FIG. 2j). We therefore use the term DISCOVER-Seq+ to denote CRISPR off-target discovery that combines MRE11 ChIP-seq (i.e., DISCOVER-Seq) with DNA-PKcs inhibition to achieve improved detection sensitivity.

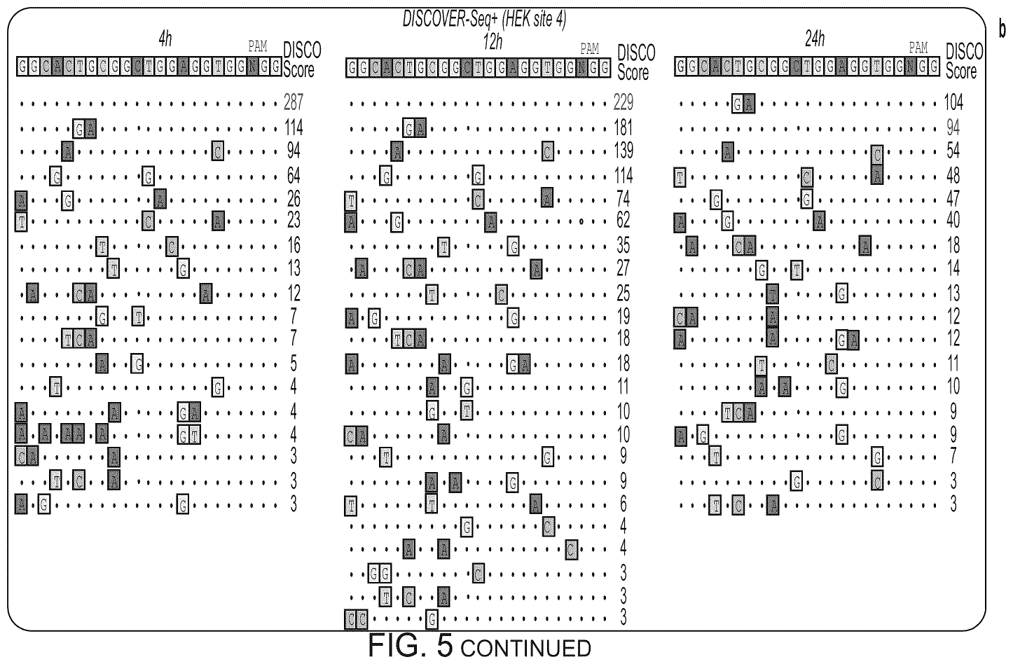

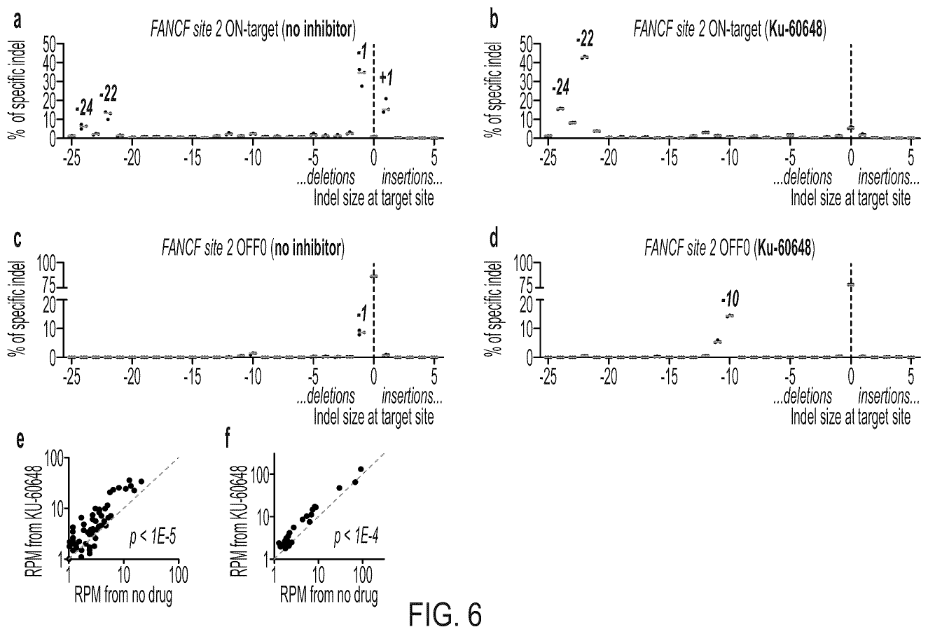

Next, we compared the results of DISCOVER-Seq+ to alternative methods for measuring off-target CRISPR activity. Targeted deep sequencing can directly measure the level of genome editing outcomes at putative off-target sites, but is limited by lower sensitivity because it relies on detection of indel editing products [28]. For the FANCF site 2 gRNA, select off-target sites exclusively detected by DISCOVER-Seq+ (and not by DISCOVER-Seq) indeed exhibited insertion-deletion mutations (indels) after 4 days as measured by targeted deep sequencing of cells exposed to Cas9 but without DNA-PKcs inhibition (FIG. 2k). Cells with DNA-PKcs inhibition exhibited indels with greater deletions, consistent with modulation of repair to the MMEJ pathway (FIG. 6a-d). The majority of discovered off-target sites were also found by GUIDE-seq, an alternate method for off-target detection that is not amenable to in vivo systems (FIG. 2l) [8,13]. Many FANCF site 2 DISCOVER-Seq+ off-target sites coincided with off-target sites independently discovered by GUIDE-seq, suggesting that the sensitivity of DISCOVER-Seq+ is comparable to GUIDE-seq and exceeds that detectable from sequencing of indel product (FIG. 2m). These results suggest that DISCOVER-Seq+ exhibits sensitivity for CRISPR off-target detection that is comparable to GUIDE-seq, and greater than targeted amplicon sequencing.

### Ex Vivo and In Vivo Applications of DISCOVER-Seq.

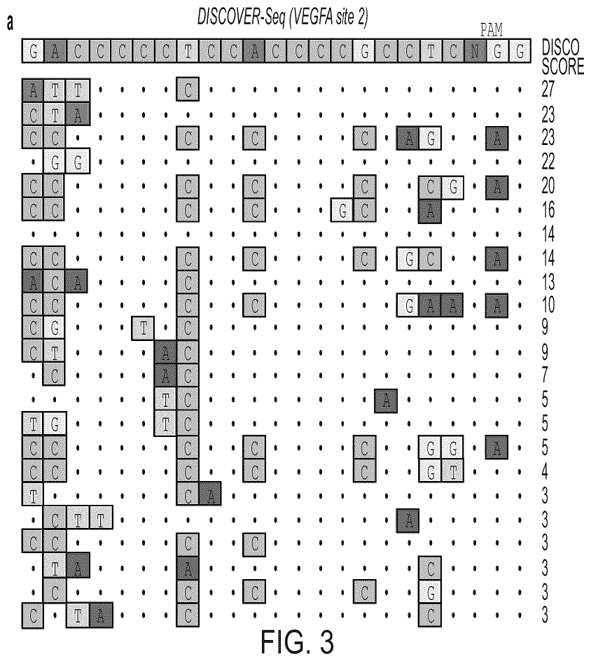

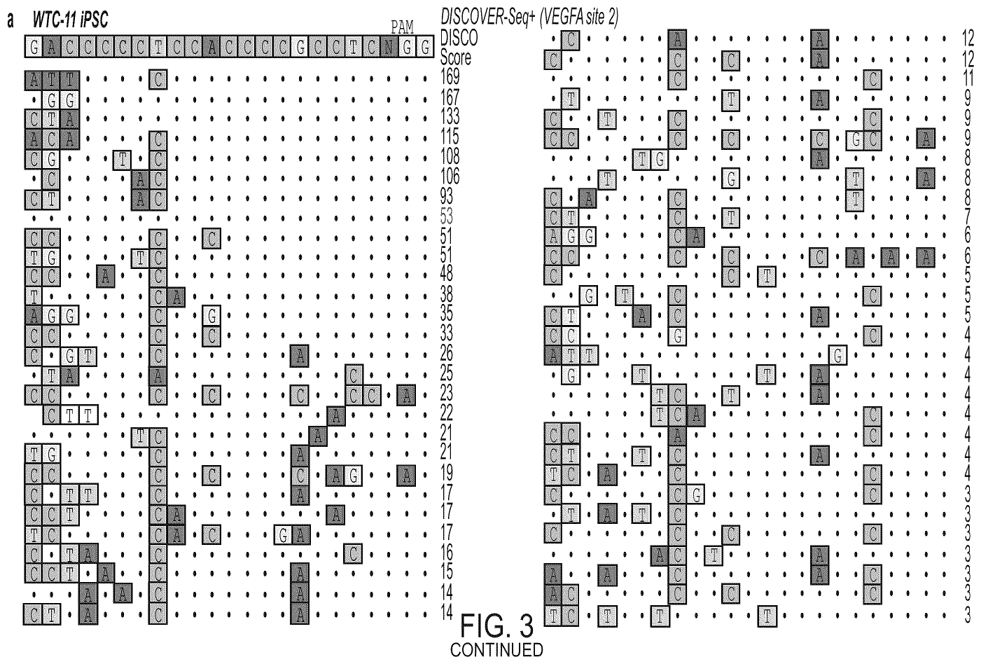

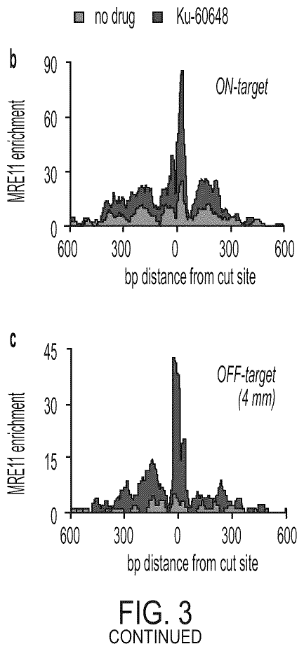

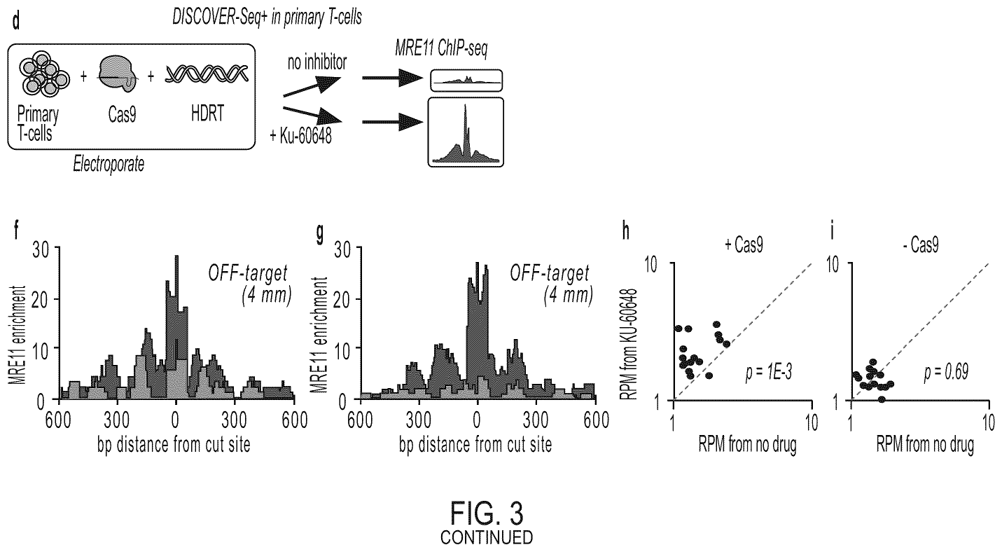

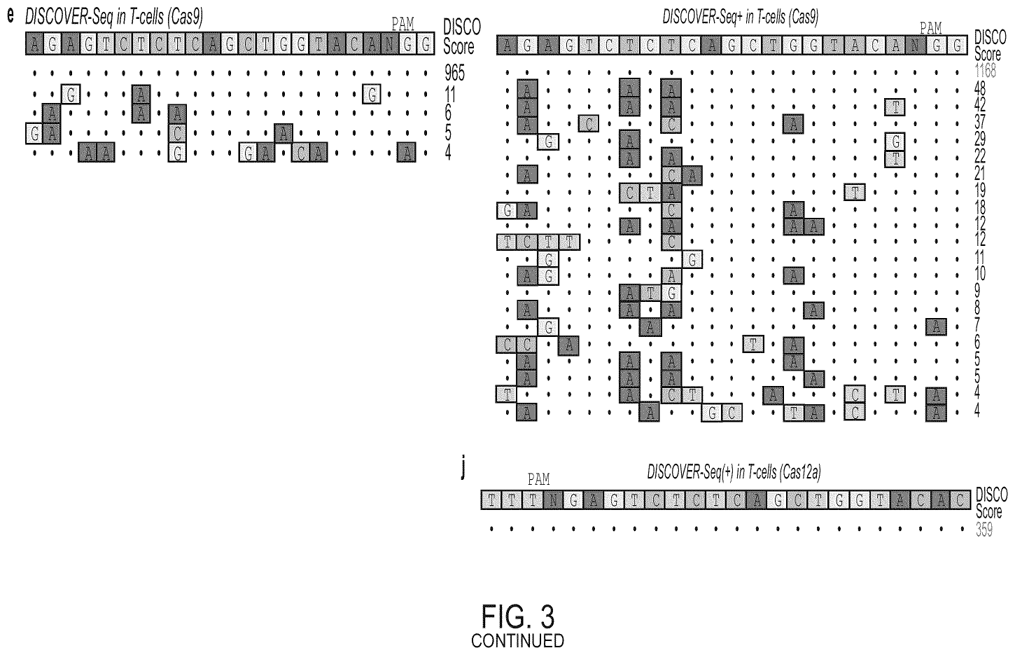

We evaluated the feasibility of DISCOVER-Seq+ in clinically translatable applications. First, we used DISCOVER-Seq+ to improve off-target detection in induced pluripotent stem cells (iPSCs). DISCOVER-Seq+ in WTC-11 iPSCs discovered over 2-fold more off-target sites at VEGFA site 2 (FIG. 3a) and led to significantly increased MRE11 ChIP-seq enrichment (p<1E-5) (FIG. 6e) compared to DISCOVER-Seq alone.

Next, we applied DISCOVER-Seq+ex vivo to identify CRISPR off-target sites during knock-in of a Chimeric antigen receptor (CAR) construct into primary human T-cells [30]. We electroporated Cas9 targeting TRA (T Cell Receptor Alpha Locus), along with a homology-directed repair template (HDRT) encoding a CAR specific for the HLA receptor loaded with a R175H mutated p53 peptide [31], then performed DISCOVER-Seq+12 hours later (FIG. 3d). DISCOVER-Seq+ (with Ku-60648) identified 20 off-target sites genome-wide compared to 4 with DISCOVER-Seq (FIG. 3e), and led to significantly greater MRE11 enrichment at all discovered sites (FIG. 3f-h). In contrast, samples without Cas9 did not exhibit any change in enrichment with Ku-60648 (FIG. 3i), further confirming that the inhibitor alone does not induce damage. We also performed the same experiment using Cas12a (Cpf1) targeting the same site in TRA. DISCOVER-Seq+ at 12 hours after Cas12a editing only identified the on-target site and no other off-target sites (FIG. 3j), consistent with the improved specificity of Cas 12a [32]. Together, these experiments demonstrate that DISCOVER-Seq+ is compatible with ex vivo editing of primary human cells, and compatible with CRISPR knock-in experiments that use a homology template.

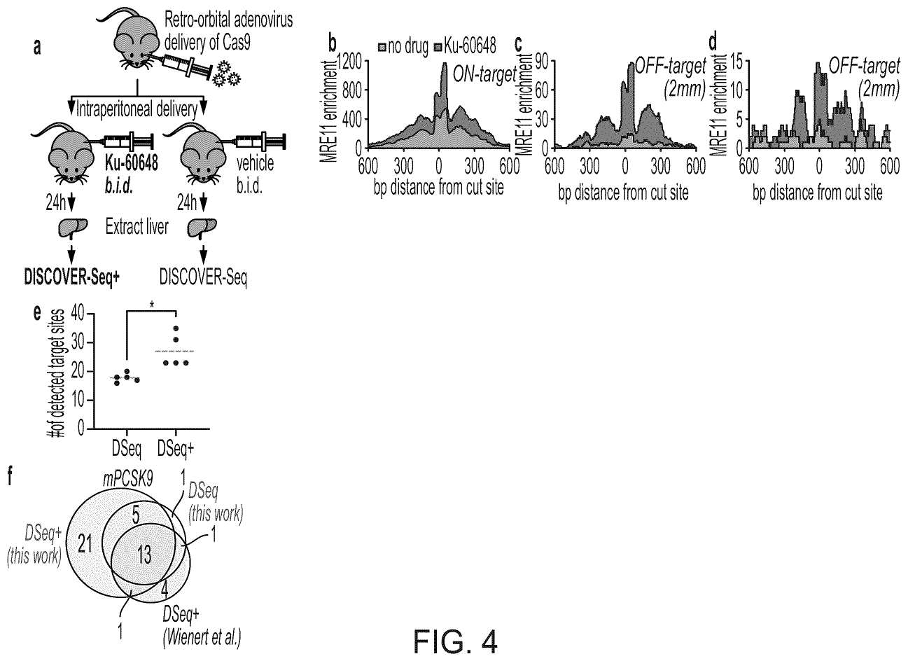

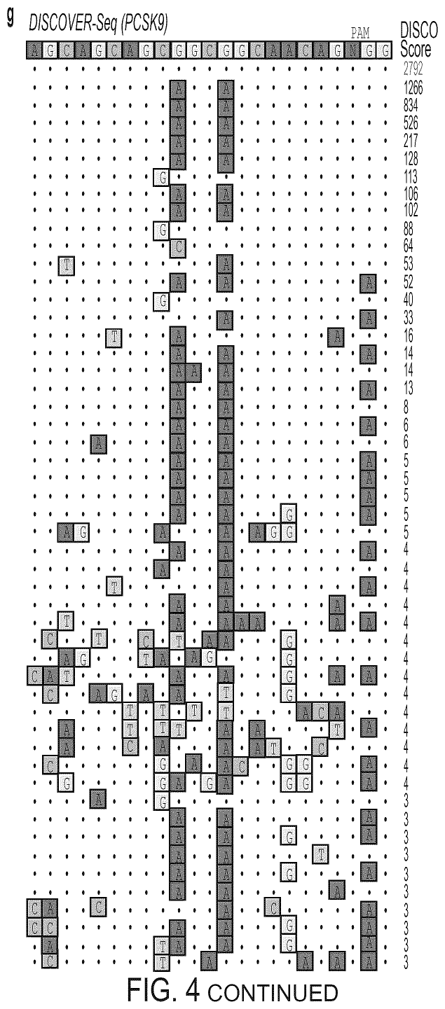

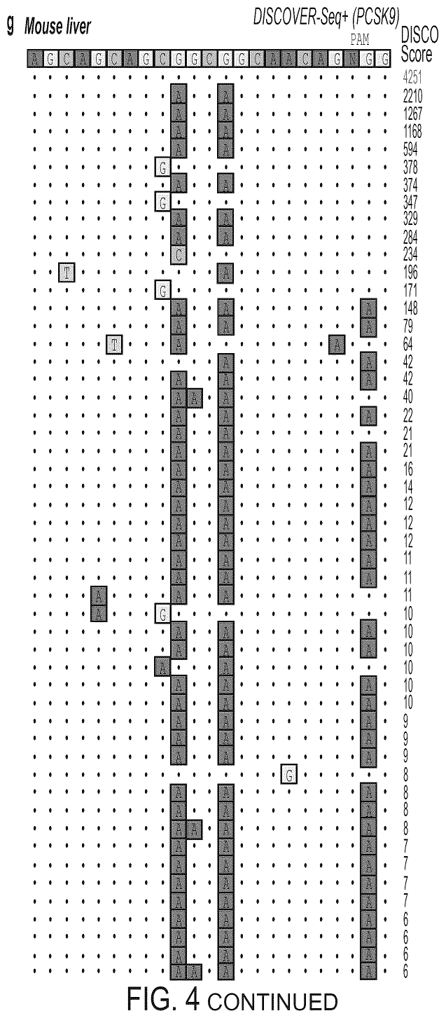

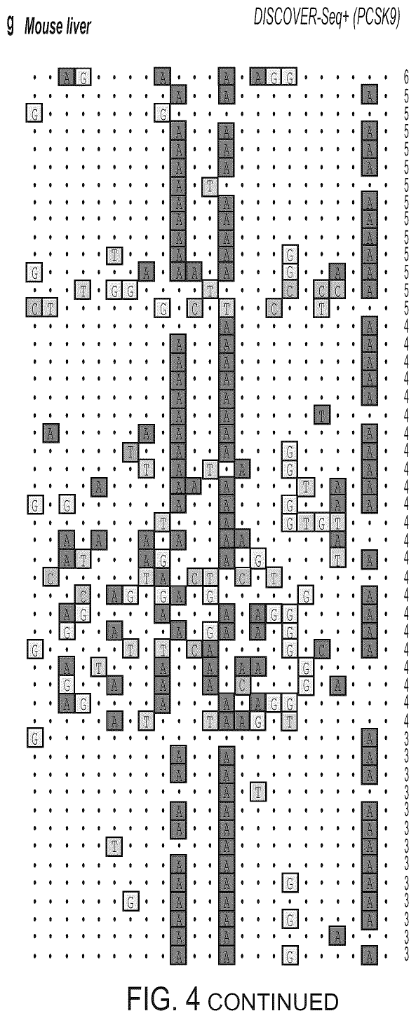

Finally, we evaluated DISCOVER-Seq+ in vivo to identify CRISPR off-target sites when targeting the cardiovascular risk allele POSK9 in mice [33]. We retro-orbitally injected adenovirus encoding Cas9 with gRNA targeting PC′SK9 into C57BL/6J mice, followed by peritoneal injection of either 25 mg/kg Ku-60648 (i.e., DISCOVER-Seq+) or vehicle (i.e., DISCOVER-Seq) twice daily (b.i.d.) (FIG. 4a). Ku-60648 has previously been evaluated as a drug for chemo-sensitization in cancer therapy, exhibits good pharmacokinetics, and strongly penetrations into tissue including tumors [21]. Mice were sacrificed after 24 hours to harvest the liver for MRE11 ChIP-seq. DISCOVER-Seq+ mice exhibited significantly increased MRE11 ChIP-seq signal in its liver compared to without DNA-PKcs inhibition (p<1E-4) (FIG. 4b-d; FIG. 6f). An average of 27 target sites were identified with DISCOVER-Seq+ compared to 18 sites with DISCOVER-Seq across 5 biological replicates (p<0.05) (FIG. 4e). The identified sites strongly overlap between the two methodologies and with sites identified in the original DISCOVER-Seq study (FIG. 4f) [13]. Pooling sequencing reads across all 5 replicates resulted in 101 target sites identified with DISCOVER-Seq+versus 51 with DISCOVER-Seq alone (FIG. 4g). Together these results demonstrate that DISCOVER-Seq+ is compatible with direct measurement of genome-wide off-target editing in vivo.

### Discussion

This study designed and validated DISCOVER-Seq+, the most sensitive method to-date for detection of CRISPR off-target activity in primary cells and in vivo. As CRISPR becomes a feasible approach for therapeutic genome editing [1,19,30,33,34], measurement of CRISPR off-target activity directly in clinically translatable applications is crucial. However, such measurements are currently challenging because the majority of off-target detection methods are limited to in vitro (e.g., CIRCLE-Seq, Digenome-seq) [4-7] or in immortalized cell lines (e.g., GUIDE-seq) [8-10], or exhibit limited sensitivity (e.g., original DISCOVER-Seq). We showed that DISCOVER-Seq+ is unique compared to previous methods by combining both high sensitivity, comparable to methods like GUIDE-Seq (FIG. 2m), with high versatility in ex vivo and in vivo applications (FIG. 3-4).

We extensively evaluated the possibility of false positive detection with DISCOVER-Seq+. False positives could theoretically arise from non-Cas9 mediated DNA damage and/or from the ChIP-seq readout itself, both of which we believe is unlikely. DNA-PKcs inhibitor is unlikely to itself induce DNA damage, because we showed that (1) cells with Ku-60648 in the absence of Cas9 had no change in the number of DNA damage foci as measured by super-resolution STED microscopy (FIG. 1f-i), (2) cells with Ku-60648 in the absence of Cas9 had no change in the level of MRE11 recruitment as measured by ChIP-seq (FIG. 3h-i), (3) in cells with Cas9, adding Ku-60648 did not increase DNA damage in regions outside of predicted off-target sites (FIG. 2e-f), and (4) off-target sites exclusively discovered by DISCOVER-Seq+ were also discovered independently using alternative methods such as GUIDE-seq (FIG. 2m). It is also unlikely for false positives to arise from the ChIP-seq readout itself because (1) MRE11 enrichment for samples with Cas9 are always normalized by a no-Cas9 negative control, (2) only genomic locations with fewer than 8 mismatches from the gRNA are even considered [13], and (3) any putative off-target sites also identified in the no-Cas9 negative control are excluded, and (4) DISCOVER-Seq, which also uses MRE11 ChIP-seq [13], has been reported to have minimal false positives [35].

DISCOVER-Seq+, like other off-target detection methods, identified off-target sites that did not have detectable indel mutations using deep amplicon sequencing (FIG. 2m) [8,35]. DISCOVER-Seq+ directly measures off-target DNA damage by measuring the levels of a DNA repair protein, in notable contrast to measurement of off-target mutagenesis with amplicon sequencing. For off-target detection, DNA damage is the most relevant measurement because the fundamental question is whether CRISPR exerts cleavage activity at unintended genomic sites, not necessarily whether the DNA eventually mutates. Furthermore, indels comprises only a subset of DNA damage outcomes, and other mutations, such as large deletions and translocations [8,36], may be missed by deep amplicon sequencing. In summary, measurement of off-target DNA damage with DISCOVER-Seq+ directly measures the parameter of interest-genome-wide CRISPR cleavage- and absence of detectable indels at select off-target sites does not reflect a false positive measurement.

In conclusion, identifying off-target genome editing in therapeutically relevant systems is a major barrier to clinical translation of CRISPR systems. By leveraging MRE11 ChIP-seq with DNA-PKcs inhibition, DISCOVER-Seq+ provides the highest detection sensitivity to date in versatile systems from ex vivo editing of primary human cells to in vivo editing of mice, setting the standard for genome-wide CRISPR off-target discovery. DISCOVER-Seq+ has the potential to validate the specificity profile of genome editing at numerous stages of the therapeutic development pipeline from cell lines and primary cells to mice and potentially non-human primates [1,17,30,33,34].

### REFERENCES

1. Doudna, J. A. The promise and challenge of therapeutic genome editing. Nature 578, 229-236 (2020).
2. Yeh, C. D., Richardson, C. D. & Corn, J. E. Advances in genome editing through control of DNA repair pathways. Nature Cell Biology 21, 1468-1478 (2019).
3. Tsai, S. Q. & Joung, J. K. Defining and improving the genome-wide specificities of CRISPR-Cas9 nucleases. Nature Reviews Genetics 17, 300-312 (2016).
4. Kim, D. et al. Digenome-seq: genome-wide profiling of CRISPR-Cas9 off-target effects in human cells. Nature Methods 12, 237-243 (2015).
5. Tsai, S. Q. et al. CIRCLE-seq: a highly sensitive in vitro screen for genome-wide CRISPR-Cas9 nuclease off-targets. Nature Methods 14, 607-614 (2017).
6. Kim, D. and Kim, J. S., 2018. DIG-seq: a genome-wide CRISPR off-target profiling method using chromatin DNA. Genome Research 28, 1894-1900 (2018).
7. Cameron, P. et al. Mapping the genomic landscape of CRISPR-Cas9 cleavage. Nature Methods 14, 600-606 (2017).
8. Tsai, S. Q. et al. GUIDE-seq enables genome-wide profiling of off-target cleavage by CRISPR-Cas nucleases. Nature Biotechnology 33, 187-197 (2015).
9. Yan, W. X. et al. BLISS is a versatile and quantitative method for genome-wide profiling of DNA double-strand breaks. Nature Communications 8, 1-9 (2017).
10. Frock, R. L. et al. Genome-wide detection of DNA double-stranded breaks induced by engineered nucleases. Nature Biotechnology 33, 179-186 (2015).
11. Singh, D., Sternberg, S. H., Fei, J., Doudna, J. A. and Ha, T. Real-time observation of DNA recognition and rejection by the RNA-guided endonuclease Cas9. Nature Communications 7, 1-8 (2016).
12. Schep, R. et al. Impact of chromatin context on Cas9-induced DNA double-strand break repair pathway balance. Molecular Cell 81, 2216-2230 (2021).
13. Wienert, B. et al. Unbiased detection of CRISPR off-targets in vivo using DISCOVER-Seq. Science 364, 286-289 (2019).
14. Akcakaya, P. et al. In vivo CRISPR editing with no detectable genome-wide off-target mutations. Nature 561, 416-419 (2018).
15. Liang, S. Q. et al. Genome-wide detection of CRISPR editing in vivo using GUIDE-tag. Nature Communications 13, 1-14 (2022).
16. Liu, Y. et al. Very fast CRISPR on demand. Science 368, 1265-1269 (2020).
17. Zou, R. S., Liu, Y., Wu, B. & Ha, T. Cas9 deactivation with photocleavable guide RNAs. Molecular Cell 81, 1553-1565 (2021).
18. Cano, C. 1-Substituted (Dibenzo [b, d]thiophen-4-yl)-2-morpholino-4 H-chromen-4-ones Endowed with Dual DNA-PK/PI3-K Inhibitory Activity. Journal of Medicinal Chemistry 56, 6386-6401 (2013).
19. Mullard, A. Gene-editing pipeline takes off. Nature Reviews Drug Discovery 19, 367-373 (2020).
20. Scully, R., Panday, A., Elango, R. & Willis, N. A. DNA double-strand break repair-pathway choice in somatic mammalian cells. Nature Reviews Molecular Cell Biology 20, 698-714 (2019).
21. Munck, J. M. et al. Chemosensitization of cancer cells by KU-0060648, a dual inhibitor of DNA-PK and PI-3K. Molecular Cancer Therapeutics 11, 1789-1798 (2012).
22. Nutley, B. P. et al. Preclinical pharmacokinetics and metabolism of a novel prototype DNA-PK inhibitor NU7026. British Journal of Cancer 93, 1011-1018 (2005).
23. Han, K. Y. and Ha, T., 2015. Dual-color three-dimensional STED microscopy with a single high-repetition-rate laser. Optics letters, 40(11), pp. 2653-2656.
24. Ma, Y. and Ha, T., 2019. Fight against background noise in stimulated emission depletion nanoscopy. Physical biology, 16(5), p. 051002.
25. Isono, M. et al. BRCA1 directs the repair pathway to homologous recombination by promoting 53BP1 dephosphorylation. Cell Reports 18, 520-532 (2017).
26. Zhong, Q., Chen, C. F., Chen, P. L. & Lee, W. H. BRCA1 facilitates microhomology-mediated end joining of DNA double strand breaks. Journal of Biological Chemistry 277, 28641-28647 (2002).
27. Zou, R. S. et al. Massively parallel genomic perturbations with multi-target CRISPR interrogates Cas9 activity and DNA repair at endogenous sites. Nature Cell Biology 24, 1433-1444 (2022).
28. Pinello, L. et al. Analyzing CRISPR genome-editing experiments with CRISPResso. Nature Biotechnology 34, 695-697 (2016).
29. Kreitzer, F. R. et al. A robust method to derive functional neural crest cells from human pluripotent stem cells. American Journal of Stem Cells 2, 119 (2013).
30. June, C. H., O'Connor, R. S., Kawalekar, O. U., Ghassemi, S. and Milone, M. C. CAR T cell immunotherapy for human cancer. Science 359, 1361-1365 (2018).
31. Chiang, Y. T. et al. The function of the mutant p53-R175H in cancer. Cancers 13, 4088 (2021).
32. Kleinstiver, B. P. et al. Genome-wide specificities of CRISPR-Cas Cpf1 nucleases in human cells. Nature Biotechnology 34, 869-874 (2016).
33. Musunuru, K. In vivo CRISPR base editing of PCSK9 durably lowers cholesterol in primates. Nature 593, 429-434 (2021).
34. Stadtmauer, E. A. et al. CRISPR-engineered T cells in patients with refractory cancer. Science 367, 7365 (2020).
35. Bao, X. R., Pan, Y., Lee, C. M., Davis, T. H. and Bao, G. Tools for experimental and computational analyses of off-target editing by programmable nucleases. Nature Protocols 16, 10-26 (2021).
36. Kosicki, M., Tomberg, K. and Bradley, A. Repair of double-strand breaks induced by CRISPR-Cas9 leads to large deletions and complex rearrangements. Nature Biotechnology 36, 765-771 (2018).

### OTHER EMBODIMENTS

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. A method of detecting off-target genome editing in vitro or in vivo, comprising:
contacting a cell in vitro or in vivo with a Clustered Regularly Interspaced Short Palindromic Repeat (CRISPR)-associated endonuclease and at least one guide RNA (gRNA) that targets a nucleic acid sequence of interest;
administering to a cell in vitro or in vivo an inhibitor of DNA repair, wherein the inhibitor modulates meiotic recombination 11 (MRE11) localization at the target nucleic acid sequence;
detecting MRE11 at on- and off-target nucleic acid sequences;
thereby detecting off-target genome editing in vitro or in vivo.

2. The method of claim 1, wherein the MRE11 is detected by chromatin immunoprecipitation with sequencing (ChIP-seq) and/or quantified by qPCR.

3. The method of claim 1, wherein the inhibitor of DNA repair comprises one or more DNA-dependent protein kinase catalytic subunit (DNA-PKcs) inhibitors.

4. The method of claim 3, wherein the one or more DNA-Pkc inhibitors comprise 2-(4-ethylpiperazin-1-yl)-N-[4-(2-morpholin-4-yl-4-oxochromen-8-yl)dibenzothiophen-1-yl]acetamide (Ku-60648), 2-(morpholin-4-yl)benzo (h) chromen-4-one (Nu7026), N-methyl-2-morpholin-4-yl-N-[6-[2-(8-oxa-3-azabicyclo[3.2.1]octan-3-yl)-4-oxochromen-8-yl]dibenzothiophen-2-yl]acetamide (NU5455), 7-methyl-2-[(7-methyl-[1,2,4]triazolo[1,5-a]pyridin-6-yl)amino]-9-(oxan-4-yl) purin-8-one (AZD7648), 2-N-morpholino-8-dibenzofuranyl-chromen-4-one (NU7427), 2-N-morpholino-8-dibenzothiophenyl-chromen-4-one (NU7441), non-coding microRNAs (miRNAs), siRNAs or combinations thereof.

5. The method of claim 4, wherein the DNA-Pkc inhibitor is Ku-60648.

6. The method of claim 4, wherein the DNA-Pkc inhibitor is Nu7026.

7. The method of claim 1, wherein the CRISPR-associated endonuclease is Type I, Type II, or Type III Cas endonuclease.

8. The method of claim 1, wherein the CRISPR-associated endonuclease is a Cas9 endonuclease, a Cas12 endonuclease, a CasX endonuclease, or a CasΦ endonuclease.

9. The method of claim 1, wherein the CRISPR-associated endonuclease is a Cas9 nuclease.

10. (canceled)

11. A method of determining specificity of a candidate genome editing complex in vitro or a subject in vivo, comprising:
contacting a cell in vitro or in vivo with a Clustered Regularly Interspaced Short Palindromic Repeat (CRISPR)-associated endonuclease and at least one guide RNA (gRNA) that targets a nucleic acid sequence of interest;
administering to a cell in vitro or in vivo an inhibitor of DNA repair, wherein the inhibitor modulates meiotic recombination 11 (MRE11) localization at the target nucleic acid sequence;
detecting MRE11 at on- and off-target nucleic acid sequences;
thereby determining specificity of a candidate genome editing complex.

12. (canceled)

13. The method of claim 11, wherein the inhibitor of DNA repair comprises one or more DNA-dependent protein kinase catalytic subunit (DNA-PKcs) inhibitors.

14. The method of claim 13, wherein the one or more DNA-Pkc inhibitors comprise 2-(4-ethylpiperazin-1-yl)-N-[4-(2-morpholin-4-yl-4-oxochromen-8-yl)dibenzothiophen-1-yl]acetamide (Ku-60648), 2-(morpholin-4-yl)benzo (h) chromen-4-one (Nu7026), N-methyl-2-morpholin-4-yl-N-[6-[2-(8-oxa-3-azabicyclo[3.2.1]octan-3-yl)-4-oxochromen-8-yl]dibenzothiophen-2-yl]acetamide (NU5455), 7-methyl-2-[(7-methyl-[1,2,4]triazolo[1,5-a]pyridin-6-yl)amino]-9-(oxan-4-yl) purin-8-one (AZD7648), 2-N-morpholino-8-dibenzofuranyl-chromen-4-one (NU7427), 2-N-morpholino-8-dibenzothiophenyl-chromen-4-one (NU7441), non-coding microRNAs (miRNAs), siRNAs or combinations thereof.

15. The method of claim 11 wherein the CRISPR-associated endonuclease is Type I, Type II, or Type III Cas endonuclease.

16. The method of claim 11, wherein the CRISPR-associated endonuclease is a Cas9 endonuclease, a Cas12 endonuclease, a CasX endonuclease, or a CasΦ endonuclease.

17. The method of claim 11, wherein the CRISPR-associated endonuclease is a Cas9 nuclease.

18. The method of claim 11, further comprising extracting a sample from the subject.

19. The method of claim 11, wherein the MRE11 is detected by chromatin immunoprecipitation with sequencing (ChIP-seq) and/or quantification by qPCR.

20. The method of claim 11, wherein the sequence of the on- and off-target nucleic acid sequences is detected by conducting high-throughput sequencing, sequencing, chromatin immunoprecipitation with sequencing (ChIP-seq) and/or quantification by qPCR.

21. A method of determining specificity of a gene editing complex, comprising:
contacting a cell with a gene-editing complex and at least one guide RNA (gRNA) that targets a nucleic acid sequence of interest;
administering to a cell an inhibitor of DNA repair;
assaying for target nucleic acid sequences comprising one or more nicks;
thereby determining specificity of a candidate genome editing complex.
The method of claim 21, wherein the gene editing complex comprises clustered regularly interspaced short palindromic repeat (CRISPR) nucleases, zinc-finger nucleases (ZFNs), transcription activator-like effector nucleases (TALENs), meganucleases, Argonaute family of endonucleases, endo- or exo-nucleases, or combinations thereof.

22. The method of claim 11 wherein the cell is contacted in vivo.
